(19) United States
(12) Patent Application Publication
(10) Pub. No.: US 2026/0028620 A1
(43) Pub. Date: Jan. 29, 2026

(54) KIT, SYSTEM, AND FLOW CELL

(71) Applicant: ILLUMINA, INC., San Diego, CA (US)

(72) Inventors: Tyler Chen, Carlsbad, CA (US); Pallavi Daggumati, San Francisco, CA (US); Tarun Kumar Khurana, Fremont, CA (US); Yir-Shyuan Wu, Albany, CA (US)

(21) Appl. No.: 19/342,340

(22) Filed: Sep. 26, 2025

Related U.S. Application Data
(62) Division of application No. 17/158,926, filed on Jan. 26, 2021.
Application No. 17/158,926 is a continuation of application No. PCT/US2021/014919, filed on Jan. 25, 2021.
(60) Provisional application No. 62/966,351, filed on Jan. 27, 2020.

(51) Int. Cl. C12N 15/10; B01J 19/00

(52) U.S. Cl. CPC C12N 15/1068; B01J 19/0046; B01J 2219/005; B01J 2219/00698; B01J 2219/00722

(57) ABSTRACT

An example of a kit includes a library preparation fluid, a sample fluid, and an enrichment fluid. The library preparation fluid includes library preparation beads, where each library preparation bead includes a first solid support, and a transposome attached to the first solid support. The fluid includes a genomic deoxyribonucleic acid sequence. The enrichment fluid includes target capture beads, where each target capture bead includes a second solid support, and capture probes attached to the second solid support. Each of the capture probes includes a single stranded deoxyribonucleic acid sequence that is complementary to a targeted region of the genomic deoxyribonucleic acid in the sample fluid.

### CROSS-REFERENCE TO RELATED APPLICATIONS

This application is a division of U.S. application Ser. No. 17/158,926, filed Jan. 26, 2021, which itself is a continuation of International Application Number PCT/US2021/014919, filed Jan. 25, 2021, which itself claims the benefit of U.S. Provisional Application Ser. No. 62/966,351, filed Jan. 27, 2020, the contents of each of which is incorporated by reference herein in its entirety.

### BACKGROUND

Flow cells are used in a variety of methods and applications, such as gene sequencing, genotyping, etc. In some methods and applications, it is desirable to generate a library of fragmented and tagged deoxyribonucleic acid (DNA) molecules from double-stranded DNA (dsDNA) target molecules. Often, the purpose is to generate smaller DNA molecules (e.g., DNA fragments) from larger dsDNA molecules for use as templates in DNA sequencing reactions. The templates may enable short read lengths to be obtained. During data analysis, overlapping short sequence reads can be aligned to reconstruct the longer nucleic acid sequences. In some instances, pre-sequencing steps (such as barcoding of particular nucleic acid molecules) can be used to simplify the data analysis.

### INTRODUCTION

A first aspect disclosed herein is a kit, comprising: a library preparation fluid including library preparation beads, each library preparation bead including a first solid support and a transposome attached to the first solid support; a sample fluid including a genomic deoxyribonucleic acid sequence; and an enrichment fluid including target capture beads, each target capture bead including a second solid support and capture probes attached to the second solid support, each of the capture probes including a single stranded deoxyribonucleic acid sequence that is complementary to a targeted region of the genomic deoxyribonucleic acid in the sample fluid.

A second aspect disclosed herein is another kit, comprising: an RNA to cDNA conversion sub-kit; and an enrichment fluid including target capture beads, each target capture bead including a solid support and capture probes attached to the solid support, each of the capture probes including a single stranded deoxyribonucleic acid sequence that is complementary to a targeted region of cDNA generated using the RNA to cDNA conversion sub-kit.

A third aspect disclosed herein is a system, comprising: a substrate; a preparation channel defined at a first region on the substrate, the preparation channel including preparation capture sites to immobilize library preparation beads; an enrichment channel defined at a second region on the substrate downstream of the preparation channel, the enrichment channel including amplification primers and enrichment capture sites to immobilize target capture beads; and a transport channel selectively and fluidically connecting the preparation channel and the enrichment channel.

A fourth aspect disclosed herein is a flow cell, comprising: an enrichment channel defined between two surfaces, the enrichment channel including amplification primers attached to at least one of the two surfaces and target capture beads immobilized on the at least one of the two surfaces, each target capture bead including a solid support and capture probes attached to the solid support, each of the capture probes including a single stranded nucleic acid sequence that is complementary to a targeted region of a genomic deoxyribonucleic acid or of a complementary deoxyribonucleic acid.

A fifth aspect disclosed herein is a method, comprising introducing target capture beads into an enrichment channel including at least one surface containing amplification primers and enrichment capture sites, whereby at least some of the target capture beads become immobilized at the enrichment capture sites, each of the target capture beads including a solid support and capture probes attached to the solid support, each of the capture probes including a single stranded nucleic acid sequence that is complementary to a targeted region of a genomic deoxyribonucleic acid or of a complementary deoxyribonucleic acid.

It is to be understood that any features of any one of the aspects may be combined together in any desirable manner. Moreover, it is to be understood that any combination of features of the first aspect and/or of the second aspect and/or of the third aspect and/or of the fourth aspect and/or of the fifth aspect may be combined with any of the examples disclosed herein to achieve the benefits as described in this disclosure, including, for example, on flow cell enrichment.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of an example of a flow cell;

FIG. 1B is an enlarged, cross-sectional view, taken along the 1B-1B line of FIG. 1A, of an example of an enrichment channel and non-patterned sequencing surfaces;

FIG. 1C is an enlarged, cross-sectional view, taken along the 1C-1C line of FIG. 1A, of an example of an enrichment channel and patterned sequencing surfaces;

FIG. 2 is a schematic flow diagram illustrating an example library preparation method;

FIG. 3A is a schematic flow diagram illustrating another example library preparation method;

FIG. 3B is a schematic illustration of the bridges resulting from the method of FIG. 3A redrawn for clarity;

FIG. 4 is a schematic illustration of an example system including an example of a flow cell having on-board library preparation chambers;

FIG. 5 is a schematic illustration depicting an example of an enrichment method disclosed herein;

FIG. 6 is a graph depicting the relative coverage plot of target library fragments on a flow cell surface when a comparative enrichment method is used, and when an example enrichment method is used; and

FIG. 7 is a bar graph depicting the targeted cluster count for target capture beads with different probe densities.

### DETAILED DESCRIPTION

Some sequencing techniques utilize capture primers on the surface of a flow cell in order to capture and amplify DNA library fragments. Flow cell architectures and amplification processes may be utilized to generate individual monoclonal populations of amplicons of respective DNA library fragments in different areas of the flow cell. This may be desirable when analyzing all or a large portion of a DNA sample (from which the DNA library fragments are generated), as each monoclonal population can provide lots of data for each of the DNA library fragments.

In some instances, it may be desirable to analyze a target portion of the DNA sample, e.g., specific genetic variants in a given sample, as opposed to the entire sample. For this type of analysis, the target portion or region of interest may be enriched, e.g., which helps to separate the target portion from the remainder of the DNA sample. This enables the sequencing reads to be dedicated to the target portion. The enrichment technique disclosed herein utilizes on-flow cell hybridization and capture of the library fragments that correspond to the target portion.

### Definitions

Terms used herein will be understood to take on their ordinary meaning in the relevant art unless specified otherwise. Several terms used herein and their meanings are set forth below.

As used herein, the singular forms “a,” “an,” and “the” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps.

Reference throughout the specification to “one example,” “another example,” “an example,” and so forth, means that a particular element (e.g., feature, structure, composition, configuration, and/or characteristic) described in connection with the example is included in at least one example described herein, and may or may not be present in other examples. In addition, it is to be understood that the described elements for any example may be combined in any suitable manner in the various examples unless the context clearly dictates otherwise.

The terms “substantially” and “about” used throughout this disclosure, including the claims, are used to describe and account for small fluctuations, such as due to variations in processing. For example, these terms can refer to less than or equal to ±10% from a stated value, such as less than or equal to ±5% from a stated value, such as less than or equal to ±2% from a stated value, such as less than or equal to ±1% from a stated value, such as less than or equal to ±0.5% from a stated value, such as less than or equal to ±0.2% from a stated value, such as less than or equal to ±0.1% from a stated value, such as less than or equal to ±0.05% from a stated value.

Adapter A linear oligonucleotide sequence that can be fused to a nucleic acid molecule, for example, by ligation or tagmentation. The adapter may be a tag exhibiting a sequence for an intended purpose. In some examples, the adapter can exhibit a sequence that is complementary to at least a portion of a primer, for example, a primer including a universal nucleotide sequence (such as a P5 or P7 sequence). As one specific example, a transposome complex may include a polynucleotide having a 3′ portion comprising a transposon end sequence, and an adapter comprising one or more regions suitable for hybridization with a primer for a cluster amplification reaction. In another specific example, the adapter at one end of a fragment includes a sequence that is complementary to at least a portion of a first flow cell primer, and the adapter at the other end of the fragment includes a sequence that is identical to at least a portion of a second flow cell primer. The complementary adapter can hybridize to the first flow cell primer, and the identical adapter is a template for its complementary copy, which can hybridize to the second flow cell primer during clustering. In other examples, the adapter can include a sequencing primer sequence or sequencing binding site (e.g., one or more regions suitable for hybridization with a primer for a sequencing reaction). Combinations of different adapters may be incorporated into a nucleic acid molecule, such as a DNA fragment. Suitable adapter lengths may range from about 10 nucleotides to about 100 nucleotides, or from about 12 nucleotides to about 60 nucleotides, or from about 15 nucleotides to about 50 nucleotides. The adapter may include any combination of nucleotides and/or nucleic acids.

Capture Probe: A single stranded deoxyribonucleic acid sequence that is complementary to a targeted region of a genomic deoxyribonucleic acid, or of a complementary deoxyribonucleic acid derived from a ribonucleic acid sample.

Capture Site: A portion of a flow cell surface having been modified with a chemical property that allows for localization of a specific material (e.g., target capture beads, complexes, protein biomarkers, etc.). In an example, the capture site may include a chemical capture agent (i.e., a material, molecule or moiety) that is capable of attaching, retaining, or binding to a specific molecule. One example chemical capture agent includes a member of a receptor-ligand binding pair (e.g., avidin, streptavidin, biotin, lectin, carbohydrate, nucleic acid binding protein, epitope, antibody, etc.) that is capable of binding to the specific material (or to a linking moiety attached to the specific material). Yet another example of the chemical capture agent is a chemical reagent capable of forming an electrostatic interaction, a hydrogen bond, or a covalent bond (e.g., thiol-disulfide exchange, click chemistry, Diels-Alder, etc.) with the specific material. Capture sites that are designed to attach target capture beads are referred to herein as “enrichment capture sites.” Capture sites that are designed to attach solid supports for library preparation are referred to herein as “preparation capture sites.”

Depositing: Any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

Depression: A discrete concave feature in a substrate or a patterned resin having a surface opening that is at least partially surrounded by interstitial region(s) of the substrate or the patterned resin. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells. The depression may also have more complex architectures, such as ridges, step features, etc.

Each: When used in reference to a collection of items, each identifies an individual item in the collection, but does not necessarily refer to every item in the collection. Exceptions can occur if explicit disclosure or context clearly dictates otherwise.

Enrichment channel: An area defined between two bonded or otherwise attached components, which includes target capture beads immobilized therein, or can receive and immobilize target capture beads therein. In some examples, the enrichment channel may be defined between two patterned or non-patterned sequencing surfaces, and thus may be in fluid communication with one or more components of the sequencing surfaces.

Flow Cell: A vessel having a chamber (e.g., which may include an enrichment channel) where a reaction can be carried out, an inlet for delivering reagent(s) to the chamber, and an outlet for removing reagent(s) from the chamber. In some examples, the chamber enables the detection of the reaction that occurs in the chamber. For example, the chamber can include one or more transparent surfaces allowing for the optical detection of arrays, optically labeled molecules, or the like.

Fragment: A portion or piece of genetic material (e.g., DNA, RNA, etc.).

Nucleic acid molecule or sample: A polymeric form of nucleotides of any length, and may include ribonucleotides, deoxyribonucleotides, analogs thereof, or mixtures thereof. The term may refer to single stranded or double stranded polynucleotides.

A “template” nucleic acid molecule (or strand) may refer to a sequence that is to be analyzed.

The nucleotides in a nucleic acid sample may include naturally occurring nucleic acids and functional analogs thereof. Examples of functional analogs are capable of hybridizing to a nucleic acid in a sequence specific fashion or capable of being used as a template for replication of a particular nucleotide sequence. Naturally occurring nucleotides generally have a backbone containing phosphodiester bonds. An analog structure can have an alternate backbone linkage including any of a variety known in the art. Naturally occurring nucleotides generally have a deoxyribose sugar (e.g., found in DNA) or a ribose sugar (e.g., found in RNA). An analog structure can have an alternate sugar moiety including any of a variety known in the art. Nucleotides can include native or non-native bases. A native DNA can include one or more of adenine, thymine, cytosine and/or guanine, and a native RNA can include one or more of adenine, uracil, cytosine and/or guanine. Any non-native base may be used, such as a locked nucleic acid (LNA) and a bridged nucleic acid (BNA).

Primer A nucleic acid molecule that can hybridize to a specific sequence. As one example, an amplification primer can hybridize to an adapter attached to a library fragment, and can serve as a starting point for template amplification and cluster generation. As another example, a synthesized nucleic acid (template) strand may include a site to which a primer (e.g., a sequencing primer) can hybridize in order to prime synthesis of a new strand that is complementary to the synthesized nucleic acid strand. Any primer can include any combination of nucleotides or analogs thereof. In some examples, the primer is a single-stranded oligonucleotide or polynucleotide. The primer length can be any number of bases long and can include a variety of non-natural nucleotides. In an example, the sequencing primer is a short strand, ranging from 10 to 60 bases, or from 20 to 40 bases.

Sequencing-ready nucleic acid fragments: A portion of genetic material having adapters at the 3′ and 5′ ends. In the sequencing-ready nucleic acid fragment, each adapter includes a known universal sequence (e.g., which is complementary to or identical to at least a portion of a primer on a flow cell) and a sequencing primer sequence. Both of the adapters may also include an index (barcode or tag) sequence. In an example, one side (e.g., including a P5′ or P5 sequence) may contain a solid support index and the other side (including a P7 or P7′ sequence) may contain a sample index.

Sequencing surface: A polymeric hydrogel having one or more types of amplification primers grafted thereto. The sequencing surface may also include an enrichment capture agent to immobilize target capture beads.

Solid support: A small body made of a rigid or semi-rigid material having a shape characterized, for example, as a sphere, oval, microsphere, or other recognized particle shape whether having regular or irregular dimensions. Some examples of the solid support can have library fragments attached thereto. Other examples of the solid support can have capture probes attached thereto.

Target Capture Bead: A solid support having capture probes attached thereto.

Target Library Fragment: A single stranded portion of i) a genomic deoxyribonucleic acid sequence or ii) a complementary deoxyribonucleic acid derived from a ribonucleic acid sample. The sequence of the target library fragment is complementary to the capture probes of the target capture bead. The sequence of the target library fragment may be a region of interest, e.g., a specific genomic panel, such as exomes or tumor related genes.

Transferred Strand: A transferred portion of two transposon ends. Similarly, the term “non-transferred strand” refers to the non-transferred portion of two transposon ends. The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some examples, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the sample DNA directly by the transposition reaction, the non-transferred strand becomes attached to the DNA fragment indirectly, because the non-transferred strand is linked to the transferred strand by the loop of the hairpin structure.

Transposon End: A double-stranded nucleic acid DNA that exhibits only the nucleotide sequences (the “transposon end sequences”) that are necessary to form the complex with a transposase or integrase enzyme that is functional in an in vitro transposition reaction. In some examples, a transposon end is capable of forming a functional complex with the transposase in a transposition reaction. As examples, transposon ends can include the 19-base pair (bp) outer end (“OE”) transposon end, the inner end (“IE”) transposon end, or the “mosaic end” (“ME”) transposon end recognized by a wild-type or mutant Tn5 transposase. Transposon ends can include any nucleic acid or nucleic acid analogue suitable for forming a functional complex with the transposase or integrase enzyme in an in vitro transposition reaction. For example, the transposon end can include DNA, RNA, modified bases, non-natural bases, modified backbone, and can comprise nicks in one or both strands. Although the term “DNA” may be used throughout the present disclosure in connection with the composition of transposon ends, it should be understood that any suitable nucleic acid or nucleic acid analogue can be utilized in a transposon end.

Transposase: An enzyme that is capable of forming a functional complex with a transposon end-containing composition (e.g., transposons, transposon ends, transposon end compositions) and catalyzing insertion or transposition of the transposon end-containing composition into the double-stranded target DNA with which it is incubated, for example, in an in vitro transposition reaction. A transposase as presented herein can also include integrases from retrotransposons and retroviruses. Although many examples described herein refer to Tn5 transposase and/or hyperactive Tn5 transposase, it will be appreciated that any transposition system that is capable of inserting a transposon end with sufficient efficiency to 5′-tag and fragment a target DNA for its intended purpose can be used. In particular examples, a preferred transposition system is capable of inserting the transposon end in a random or in an almost random manner to 5′-tag and fragment the target DNA.

Transposome Complex: A transposase enzyme non-covalently bound to a double stranded nucleic acid. For example, the transposome complex can be a transposase enzyme pre-incubated with double-stranded transposon DNA under conditions that support non-covalent complex formation. Double-stranded transposon DNA can include, for example, Tn5 DNA, a portion of Tn5 DNA, a transposon end composition, a mixture of transposon end compositions or other double-stranded DNAs capable of interacting with a transposase, such as the hyperactive Tn5 transposase.

In the examples disclosed herein, target capture beads are utilized in the enrichment channel of the flow cell, which also includes sequencing surface(s). The target capture beads include capture probes, which are capable of selectively capturing library fragments of interest. Therefore, the target capture beads enable custom enrichment to take place within the flow cell.

### Target Capture Beads

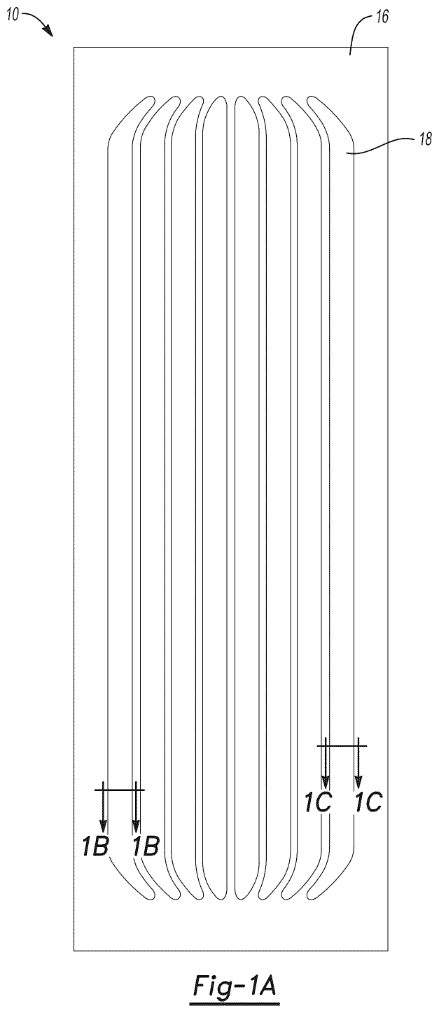

The target capture beads are shown in FIG. 1B and FIG. 1C at reference numeral 36. The target capture beads 36 include a solid support 38 and capture probes 40 attached to the solid support.

The solid support 38 may be, without limitation, glass (e.g., controlled pore glass beads); magnetically responsive materials; plastic, such as acrylic, polystyrene or a copolymer of styrene and another material, polypropylene, polyethylene, polybutylene, polyurethane or polytetrafluoroethylene (TEFLON® from The Chemours Co); polysaccharides or cross-linked polysaccharides such as agarose or SEPHAROSE® beads (cross-linked beaded form of agarose, available from Cytivia); nylon; nitrocellulose; resin; silica or silica-based materials including silicon and modified silicon; carbon-fiber; metal; inorganic glass; an optical fiber bundle; or a variety of other polymers.

A “magnetically responsive” material is responsive to a magnetic field. Examples of magnetically responsive solid supports include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe₃O₄, BaFe₁₂O₁₉, CoO, NiO, Mn₂O₃, Cr₂O₃, and CoMnP. One commercially available example includes DYNABEADS™ M-280 Streptavidin (superparamagnetic beads coated with streptavidin) from ThermoFisher Scientific. In some examples, the magnetically responsive material is embedded in the shell of a polymer bead. In other examples, the magnetically responsive material is in bead form and is coated with a passivating material, such as silicon oxide or silicon nitrite.

Any example of the solid support 38 may have the form of solid beads, porous beads, or hollow beads.

In some examples, the solid support 38 may be functionalized with one member of a binding pair. A “binding pair” refers to two agents (e.g., materials, molecules, moieties) that are capable of attaching to one another. In this example, the member on the solid support 38 is a binding pair with another member that is located on the sequencing surface (see 12, 12′ in FIG. 1B and 14, 14′ of FIG. 1C) of the flow cell 10 (FIG. 1A). In some examples, the member on the solid support 38 may be multi-functional in that it can i) bind to a member attached to the capture probes 40, and ii) bind to an enrichment capture site (see 32, 32′ in FIG. 1B and FIG. 1C) on the sequencing surface 12, 12′ or 14, 14′ of the flow cell 10. In other examples, the solid support 38 may be functionalized with two different binding pair members, e.g., i) one member that can bind to the member attached to the capture probes 40, and ii) another member that can bind to the enrichment capture site 32, 32′ on the 12, 12′ or 14, 14′ of the flow cell 10.

Functionalization of the solid support 38 may involve coating the solid support 38 with the binding pair member, or forming a bond between the binding pair member and a functional group at the surface of the solid support 38. One example of a binding pair member includes a member of a receptor-ligand binding pair (e.g., avidin, streptavidin, biotin, lectin, carbohydrate, nucleic acid binding protein, epitope, antibody, etc.) that is capable of binding to the other binding pair member that is located on the sequencing surface of the flow cell. The binding pair members may also be chemical reagents that are capable of forming an electrostatic interaction, a hydrogen bond, or a covalent bond (e.g., thiol-disulfide exchange, click chemistry, Diels-Alder, etc.).

In other examples, the solid support 38 does not include a member of a binding pair, but rather, the solid support 38 may be capable of being chemically conjugated to the enrichment capture site 32, 32′. Any form of chemical coupling may be used to attach the solid support 38 to the enrichment capture site 32, 32′.

In many instances, a reversible or cleavable interaction is desirable between the solid support 38 and the enrichment capture site 32, 32′ so that the target capture beads 36 may be removed prior to sequencing.

The target capture beads 36 also include capture probes 40 attached to the solid support 38. Each capture probe 40 on the solid support 38 includes the same single stranded deoxyribonucleic acid sequence. This sequence is selected so that each capture probe 40 selectively hybridizes to a target library fragment, which includes a sequence of interest (e.g., that is to be analyzed). In some examples, the capture probe sequence is complementary to a targeted region of the genomic deoxyribonucleic acid. In other examples, the capture probe sequence is complementary to a complementary deoxyribonucleic acid (cDNA) derived from a targeted region of a ribonucleic acid sample.

The number of nucleotides in a capture probe 40 may depend upon the number of nucleotides in the target library fragment. In some instances, the capture probe 40 is smaller than the target library fragment. The capture probe 40 may include from about 50 nucleotides to about 200 nucleotides. In one example, the capture probe 40 includes from about 80 nucleotides to about 120 nucleotides. In another example, the capture probe 40 includes 69 nucleotides.

The capture probes 40 may be prepared using any oligonucleotide synthesis technique.

One end of the capture probe 40 may be bound to the other member of the binding pair so that it can bind to the member attached to the solid support 38. For example, the capture probe 40 may be biotinylated to bind with streptavidin at the surface of the solid support 38.

The capture probes 40 may be attached to the solid support 38 by incubating the solid supports 38 in a mixture containing the capture probes 40. The capture probes 40 may be mixed into a salt buffer. Any suitable salt buffer that facilitates or enables the reaction between the capture probe 40 and the solid support 38 may be used. In one example, the salt buffer may facilitate the biotin-streptavidin reaction so that biotinylated capture probes 40 bind to the streptavidin on the solid supports 38. As examples, the salt buffer is an aqueous solution including sodium chloride, sodium citrate, or combinations thereof. In one specific example, the salt buffer includes about 0.75 M sodium chloride and about 75 mM sodium citrate in water. The number of capture probes 40 in the mixture may depend upon the number of solid supports 38 to be added to the mixture and the desired probe density for the target capture beads 36. In an example, the desired probe density of the target capture beads 36 ranges from about 2*10⁵ capture probes per solid support to about 5*10⁶ capture probes per solid support. The capture probe density of the target capture beads 36 may be controlled by adjusting a concentration of the capture probes in the mixture used for incubation.

One example method for forming the target capture beads 36 includes synthesizing the capture probes 40 with a first member of a binding pair attached to an end of each capture probe 40; and incubating the capture probes 40 with the solid support 38, wherein the solid support 38 is coated with a second member of the binding pair.

### Flow Cells

The target capture beads 36 may be used in flow cells 10 with patterned sequencing surfaces 14, 14′ or non-patterned sequencing surfaces 12, 12′. It is to be understood that the target capture beads 36 may include any example of the solid support 38 and the capture probes 40 described herein.

A top view of an example of the flow cell 10 is shown in FIG. 1A. As will be discussed in reference to FIG. 1B and FIG. 1C, some examples of the flow cell 10 include two sequencing opposed sequencing surfaces. An example of non-patterned sequencing surfaces 12, 12′ are shown in FIG. 1B, and an example of patterned sequencing surfaces 14, 14′ are shown in FIG. 1C. Each sequencing surface 12, 12′ or 14, 14′ is supported by a substrate (generally shown as 16 in FIG. 1A), and an enrichment channel (generally shown as 18 in FIG. 1A) is defined between the sequencing surfaces 12, 12′ or 14, 14′. In other examples, the flow cell 10 includes one sequencing surface supported by a substrate 16 and a lid attached to the substrate 16. In these examples, the enrichment channel 18 is defined between the sequencing surface 12 or 14 and the lid.

In any of the examples, the substrate 16 may be a single layer/material. Examples of the single layer substrate are shown at reference numeral 16A and 16A′ in FIG. 1B. Examples of suitable single layer substrates 16A, 16A′ include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

The substrate 16 may also be a multi-layered structure. Examples of the multi-layered substrate are shown at reference numeral 16B and 16B′ in FIG. 1C. Some examples of the multi-layered structure 16B, 16B′ include glass or silicon, with a coating layer of tantalum oxide or another ceramic oxide at the surface. With specific reference to FIG. 1C, other examples of the multi-layered structure 16B, 16B′ include an underlying support 20, 20′ having a patterned resin 22, 22′ thereon. Still other examples of the multi-layered substrate 16B, 16B′ may include a silicon-on-insulator (SOI) substrate.

In an example, the substrate 16 (whether single or multi-layered) may have a diameter ranging from about 2 mm to about 300 mm, or a rectangular sheet or panel having its largest dimension up to about 10 feet (˜3 meters). In an example, the substrate 16 is a wafer having a diameter ranging from about 200 mm to about 300 mm. In another example, the substrate 16 is a die having a width ranging from about 0.1 mm to about 10 mm. While example dimensions have been provided, it is to be understood that a substrate 16 with any suitable dimensions may be used. For another example, a panel may be used that is a rectangular support, which has a greater surface area than a 300 mm round wafer.

In the example shown in FIG. 1A, FIG. 1B and/or FIG. 1C, the flow cell 10 includes enrichment channels 18. While several enrichment channels 18 are shown, it is to be understood that any number of channels 18 may be included in the flow cell 10 (e.g., a single channel 18, four channels 18, etc.). In some of the examples disclosed herein, each enrichment channel 18 is an area defined between two sequencing surfaces (e.g., 12 and 12′ or 14 and 14′) and by two attached substrates (e.g., 16A and 16A′ or 16B and 16B′). In other of the examples disclosed herein, each enrichment channel 18 is an area defined between one sequencing surface (e.g., 12 or 14) and a lid. The fluids described herein can be introduced into and removed from the enrichment channel(s) 18. Each enrichment channel 18 may be isolated from each other enrichment channel 18 in a flow cell 10 so that fluid introduced into any particular enrichment channel 18 does not flow into any adjacent enrichment channel 18.

A portion of the enrichment channel 18 may be defined in the substrate 16 using any suitable technique that depends, in part, upon the material(s) of the substrate 16. In one example, a portion of the enrichment channel 18 is etched into a glass substrate 16. In another example, a portion of the enrichment channel 18 may be patterned into a resin 22, 22′ of a multi-layered substrate 16B, 16B′ using photolithography, nanoimprint lithography, etc. In still another example, a separate material (e.g., material 24 in FIG. 1B and FIG. 1C) may be applied to the substrate 16 so that the separate material defines at least a portion of the walls of the enrichment channel 18.

In an example, the enrichment channel 18 has a rectilinear configuration. The length and width of the enrichment channel 18 may be smaller, respectively, than the length and width of the substrate 16 so that portion of the substrate surface surrounding the enrichment channel 18 is available for attachment to another substrate 16. In some instances, the width of each enrichment channel 18 can be at least about 1 mm, at least about 2.5 mm, at least about 5 mm, at least about 7 mm, at least about 10 mm, or more. In some instances, the length of each enrichment channel 18 can be at least about 10 mm, at least about 25 mm, at least about 50 mm, at least about 100 mm, or more. The width and/or length of each enrichment channel 18 can be greater than, less than or between the values specified above. In another example, the enrichment channel 18 is square (e.g., 10 mm×10 mm).

The depth of each enrichment channel 18 can be as small as a few monolayers thick, for example, when microcontact, aerosol, or inkjet printing is used to deposit a separate material that defines the flow channel walls. For other examples, the depth of each enrichment channel 18 can be about 1 μm, about 10 μm, about 50 μm, about 100 μm, or more. In an example, the depth may range from about 10 μm to about 100 μm. In another example, the depth is about 5 μm or less. It is to be understood that the depth of each enrichment channel 18 be greater than, less than or between the values specified above. The depth of the enrichment channel 18 may also vary along the length and width of the flow cell 10, e.g., when a patterned sequencing surface 14, 14′ is used.

FIG. 1B illustrates a cross-sectional view of the flow cell 10 including non-patterned opposed sequencing surfaces 12, 12′. In an example, each of these surfaces 12, 12′ may be prepared on the substrate 16A, 16A′, and then the substrates 16A, 16A′ may be attached to one another to form an example of the flow cell 10. Any suitable bonding material 24, such as an adhesive, a radiation-absorbing material that aids in bonding, etc., may be used to bond the substrates 16A, 16B together.

In the example shown in FIG. 1B, a portion of the enrichment channel 18 is defined in each of the single layer substrates 16A, 16A′. For example, each substrate 16A, 16A′ may have a concave region 26, 26′ defined therein where the components of the sequencing surface 12, 12′ may be introduced. It is to be understood that any space within the concave region 26, 26′ not occupied by the components of the sequencing surface 12, 12′ may be considered to be part of the enrichment channel 18.

The sequencing surfaces 12, 12′ include a polymeric hydrogel 28, 28′, amplification primers 30, 30′ attached to the polymeric hydrogel 28, 28′, and enrichment capture sites 32, 32′.

An example of the polymeric hydrogel 28, 28′ includes an acrylamide copolymer, such as poly(N-(5-azidoacetamidylpentyl)acrylamide-co-acrylamide, PAZAM. PAZAM and some other forms of the acrylamide copolymer are represented by the following structure (I):

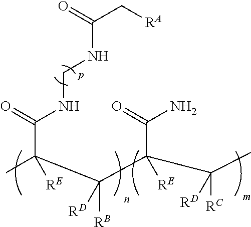

wherein:
R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa, or may be, in a specific example, about 312 kDa.

In some examples, PAZAM and other forms of the acrylamide copolymer are linear polymers. In some other examples, PAZAM and other forms of the acrylamide copolymer are a lightly cross-linked polymers.

In other examples, the polymeric hydrogel 28, 28′ may be a variation of the structure (I). In one example, the acrylamide unit may be replaced with N,N-dimethylacrylamide

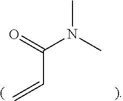

In this example, the acrylamide unit in structure (I) may be replaced with

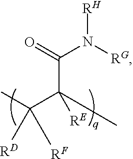

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

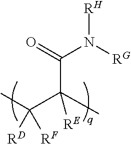

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As still another example, the polymeric hydrogel 28, 28′ may include a recurring unit of each of structure (III) and (IV):

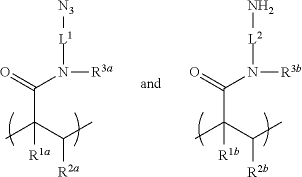

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

It is to be understood that other molecules may be used to form the polymeric hydrogel 28, 28′, as long as they are functionalized to graft oligonucleotide primers 30, 30′ thereto. Other examples of suitable polymer layers include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogels 28, 28′ include mixed copolymers of acrylamides and acrylates. A variety of polymer architectures containing acrylic monomers (e.g., acrylamides, acrylates etc.) may be utilized in the examples disclosed herein, such as branched polymers, including star polymers, star-shaped or star-block polymers, dendrimers, and the like. For example, the monomers (e.g., acrylamide, etc.) may be incorporated, either randomly or in block, into the branches (arms) of a star-shaped polymer.

To introduce the polymeric hydrogel 28, 28′ into the concave regions 26, 26′, a mixture of the polymeric hydrogel 28, 28′ may be generated and then applied to the respective substrates 16A, 16A′ (having the concave regions 26, 26′ defined therein). In one example, the polymeric hydrogel 28, 28′ may be present in a mixture (e.g., with water or with ethanol and water). The mixture may then be applied to the respective substrate surfaces (including in the concave regions 26, 26′) using spin coating, or dipping or dip coating, or flow of the material under positive or negative pressure, or another suitable technique. These types of techniques blanketly deposit the polymeric hydrogel 28, 28′ on the substrate respective substrates 16A, 16A′ (e.g., in the concave regions 26, 26′ and on interstitial regions 34, 34′ adjacent thereto). Other selective deposition techniques (e.g., involving a mask, controlled printing techniques, etc.) may be used to specifically deposit the polymeric hydrogel 28, 28′ in the concave regions 26, 26′ and not on the interstitial regions 34, 34′.

In some examples, the substrate surface (including the concave regions 26, 26′) may be activated, and then the mixture (including the polymeric hydrogel 28, 28′) may be applied thereto. In one example, a silane or silane derivative (e.g., norbornene silane) may be deposited on the substrate surface using vapor deposition, spin coating, or other deposition methods. In another example, the substrate surface may be exposed to plasma ashing to generate surface-activating agent(s) (e.g., —OH groups) that can adhere to the polymeric hydrogel 28, 28′.

Depending upon the chemistry of the polymeric hydrogel 28, 28′, the applied mixture may be exposed to a curing process. In an example, curing may take place at a temperature ranging from room temperature (e.g., about 25° C.) to about 95° C. for a time ranging from about 1 millisecond to about several days.

Polishing may then be performed in order to remove the polymeric hydrogel 28, 28′ from the interstitial regions 34, 34′ at the perimeter of the concave regions 26, 26′, while leaving the polymeric hydrogel 28, 28′ on the surface in the concave regions 26, 26′ at least substantially intact.

The sequencing surfaces 12, 12′ also include amplification primers 30, 30′ attached to the polymeric hydrogel 28, 28′.

A grafting process may be performed to graft the amplification primers 30, 30′ to the polymeric hydrogel 28, 28′ in the concave regions 26, 26′. In an example, the amplification primers 30, 30′ can be immobilized to the polymeric hydrogel 28, 28′ by single point covalent attachment at or near the 5′ end of the primers 30, 30′. This attachment leaves i) an adapter-specific portion of the primers 30, 30′ free to anneal to its cognate sequencing-ready nucleic acid fragment and ii) the 3′ hydroxyl group free for primer extension. Any suitable covalent attachment may be used for this purpose. Examples of terminated primers that may be used include alkyne terminated primers (e.g., which may attach to an azide surface moiety of the polymeric hydrogel 28, 28′), or azide terminated primers (e.g., which may attach to an alkyne surface moiety of the polymeric hydrogel 28, 28′).

Specific examples of suitable primers 30, 30′ include P5 and P7 primers used on the surface of commercial flow cells sold by Illumina Inc. for sequencing on HISEQ™, HISEQX™, MISEQ™, MISEQDX™, MINISEQ™, NEXTSEQ™, NEXTSEQDX™, NOVASEQ™, GENOME ANALYZER™, ISEQ™, and other instrument platforms. Both P5 and P7 primers may be grafted to each of the polymeric hydrogels 28, 28′.

In an example, grafting may involve flow through deposition (e.g., using a temporarily bound lid), dunk coating, spray coating, puddle dispensing, or by another suitable method that will attach the primer(s) 30, 30′ to the polymeric hydrogel 28, 28′. Each of these example techniques may utilize a primer solution or mixture, which may include the primer(s) 30, 30′, water, a buffer, and a catalyst. With any of the grafting methods, the primers 30, 30′ react with reactive groups of the polymeric hydrogel 28, 28′ in the concave region 26, 26′ and have no affinity for the surrounding substrate 16A, 16A′. As such, the primers 30, 30′ selectively graft to the polymeric hydrogel 28, 28′.

In the example shown in FIG. 1B, the enrichment capture site 32, 32′ includes a chemical capture agent that is attached to or applied on at least a portion of the polymeric hydrogel 28, 28′. Any example of the chemical capture agent disclosed herein may be used. In some examples, the chemical capture agent may be biotin, which can attach streptavidin-containing target capture beads 36 to the flow cell sequencing surface(s) 12, 12′ or 14, 14′. In other examples, the chemical capture agent may be another member of a binding pair (e.g., other than the biotin-streptavidin binding pair). In some of these other examples, the target capture beads 36 may include two different binding pair members, e.g., i) streptavidin (which binds to biotin to attach the capture probes 40), and ii) another member, which can bind to the chemical capture agent of the enrichment capture site 32, 32′ on the sequencing surface 12, 12′ or 14, 14′ of the flow cell 10. In these other examples, the chemical capture agent may be a non-biotin member of a binding pair, where the other member of the binding pair is attached to the solid support 38 in addition to the streptavidin.

In some examples, free functional groups (e.g., those not attached to primers 30, 30′) of the polymeric hydrogel 28, 28′ may be functionalized with the chemical capture agent so that several enrichment capture sites 32, 32′ are formed across the surface of the polymeric hydrogel 28, 28′. In an example, alkyne-PEG-biotin linkers or alkyne-biotin free azide groups may be covalently attached to free azides on the polymeric hydrogel 28, 28′ using click chemistry. In other examples, primers that are complementary to the amplification primers 30, 30′ may have the chemical capture agent (e.g., biotin or another member of a binding pair) attached thereto. These complementary primers may be hybridized to some of the amplification primers 30, 30′ to form the enrichment capture sites 32, 32′.

In another example, the chemical capture agent may be deposited in a desirable location using microcontact printing, aerosol printing, etc. to form the enrichment capture site(s) 32, 32′. In still another example, a mask (e.g., a photoresist) may be used to define the space/location where the chemical capture agent will be deposited, and thus where the enrichment capture site 32, 32′ will be formed. The chemical capture agent may then be deposited, and the mask removed (e.g., via lift-off, dissolution, or another suitable technique). In this example, the enrichment capture site 32, 32′ may include a monolayer or thin layer of the chemical capture agent.

FIG. 1C illustrates a cross-sectional view of the flow cell 10 including patterned opposed sequencing surfaces 14, 14′. In an example, each of these surfaces 14, 14′ may be prepared on the substrate 16B, 16B′, and then the substrates 16B, 16B′ may be attached to one another (e.g., via material 24) to form an example of the flow cell 10.

In the example shown in FIG. 1C, the flow cell 10 includes the multi-layer substrate 16B, 16B′, each of which includes the support 20, 20′ and the patterned material 22, 22′ positioned on the support 20, 20′. The patterned material 22, 22′ defines depressions 42, 42′ separated by interstitial regions 34, 34′.

In the example shown in FIG. 1C, the patterned material 22, 22′ is respectively positioned on the support 20, 20′. It is to be understood that any material that can be selectively deposited, or deposited and patterned to form the depressions 42, 42′ and the interstitial regions 34, 34′ may be used for the patterned material 22, 22′.

As one example, an inorganic oxide may be selectively applied to the support 20, 20′ via vapor deposition, aerosol printing, or inkjet printing. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

As another example, a resin may be applied to the support 20, 20′ and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, printing techniques, etc. Some examples of suitable resins include a polyhedral oligomeric silsesquioxane resin (commercially available under the tradename POSS® from Hybrid Plastics)-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” refers to a chemical composition that is a hybrid intermediate (e.g., RSiO_{1.5}) between that of silica (SiO₂) and silicone (R₂SiO). An example of a polyhedral oligomeric silsesquioxane can be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups. The resin composition disclosed herein may comprise one or more different cage or core structures as monomeric units.

As shown in FIG. 1C, the patterned material 22, 22′ includes the depressions 42, 42′ respectively defined therein, and interstitial regions 34, 34′ separating adjacent depressions 42, 42′. Many different layouts of the depressions 42, 42′ may be envisaged, including regular, repeating, and non-regular patterns. In an example, the depressions 42, 42′ are disposed in a hexagonal grid for close packing and improved density. Other layouts may include, for example, rectilinear (rectangular) layouts, triangular layouts, and so forth. In some examples, the layout or pattern can be an x-y format of depressions 42, 42′ that are in rows and columns. In some other examples, the layout or pattern can be a repeating arrangement of depressions 42, 42′ and/or interstitial regions 34, 34′. In still other examples, the layout or pattern can be a random arrangement of depressions 42, 42′ and/or interstitial regions 34, 34′. The pattern may include wells, stripes, swirls, lines, triangles, rectangles, circles, arcs, checks, plaids, diagonals, arrows, squares, and/or cross-hatches.

The layout or pattern of the depressions 42, 42′ may be characterized with respect to the density of the depressions 42, 42′ (e.g., number of depressions 42, 42′) in a defined area. For example, the depressions 42, 42′ may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density of depressions 42, 42′ in the patterned material 22, 22′ can be between one of the lower values and one of the upper values selected from the ranges above. As examples, a high density array may be characterized as having depressions 42, 42′ separated by less than about 100 nm, a medium density array may be characterized as having depressions 42, 42′ separated by about 400 nm to about 1 μm, and a low density array may be characterized as having depressions 42, 42′ separated by greater than about 1 μm. While example densities have been provided, it is to be understood that any suitable densities may be used. The density of the depressions 42, 42′ may depend, in part, on the depth of the depressions 42, 42′. In some instances, it may be desirable for the spacing between depressions 42, 42′ to be even greater than the examples listed herein.

The layout or pattern of the depressions 42, 42′ may also or alternatively be characterized in terms of the average pitch, or the spacing from the center of the depression 42, 42′ to the center of an adjacent depression 42, 42′ (center-to-center spacing) or from the right edge of one depression 42, 42′ to the left edge of an adjacent depression 42, 42′ (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of depressions 42, 42′ can be between one of the lower values and one of the upper values selected from the ranges above. In an example, the depressions 42, 42′ have a pitch (center-to-center spacing) of about 1.5 μm. While example average pitch values have been provided, it is to be understood that other average pitch values may be used.

The size of each depression 42, 42′ may be characterized by its volume, opening area, depth, and/or diameter.

Each depression 42, 42′ can have any volume that is capable of confining at least some fluid that is introduced into the flow cell 10. The minimum or maximum volume can be selected, for example, to accommodate the throughput (e.g., multiplexity), resolution, nucleotides, or analyte reactivity expected for downstream uses of the flow cell 10. For example, the volume can be at least about 1×10⁻³ μm³, at least about 1×10⁻² μm³, at least about 0.1 μm³, at least about 1 μm³, at least about 10 μm³, at least about 100 μm³, or more. Alternatively or additionally, the volume can be at most about 1×10⁴ μm³, at most about 1×10³ μm³, at most about 100 μm³, at most about 10 μm³, at most about 1 μm³, at most about 0.1 μm³, or less.

The area occupied by each depression opening can be selected based upon similar criteria as those set forth above for the volume. For example, the area for each depression opening can be at least about 1×10⁻³ μm², at least about 1×10⁻² μm², at least about 0.1 μm², at least about 1 μm², at least about 10 μm², at least about 100 μm², or more. Alternatively or additionally, the area can be at most about 1×10³ μm², at most about 100 μm², at most about 10 μm², at most about 1 μm², at most about 0.1 μm², at most about 1×10⁻² μm², or less. The area occupied by each depression opening can be greater than, less than or between the values specified above.

The depth of each depression 42, 42′ can be large enough to house some of the polymeric hydrogel 28, 28′. In an example, the depth may be at least about 0.1 μm, at least about 0.5 μm, at least about 1 μm, at least about 10 μm, at least about 100 μm, or more. Alternatively or additionally, the depth can be at most about 1×10³ μm, at most about 100 μm, at most about 10 μm, or less. In some examples, the depth is about 0.4 μm. The depth of each depression 42, 42′ can be greater than, less than or between the values specified above.

In some instances, the diameter or length and width of each depression 42, 42′ can be at least about 50 nm, at least about 0.1 μm, at least about 0.5 μm, at least about 1 μm, at least about 10 μm, at least about 100 μm, or more. Alternatively or additionally, the diameter or length and width can be at most about 1×10³ μm, at most about 100 μm, at most about 10 μm, at most about 1 μm, at most about 0.5 μm, at most about 0.1 μm, or less (e.g., about 50 nm). In some examples, the diameter is or each of the length and the width is about 0.4 μm. The diameter or length and width of each depression 42, 42′ can be greater than, less than or between the values specified above.

In this example, at least some of components of the sequencing surface 14, 14′ may be introduced into the depressions 42, 42′. It is to be understood that any space within the depressions 42, 42′ not occupied by the components of the sequencing surface 14, 14′ may be considered to be part of the enrichment channel 18.

In the example shown in FIG. 1C, the polymeric hydrogel 28, 28′ is positioned within each of the depressions 42, 42′. The polymeric hydrogel 28, 28′ may be applied as described in reference to FIG. 1B, so that the polymeric hydrogel 28, 28′ is present in the depressions 42, 42′ and not present on the surrounding interstitial regions 34, 34′ or enrichment capture site(s) 32, 32′.

In the example shown in FIG. 1C, the primers 30, 30′ may be grafted to the polymeric hydrogel 28, 28′ within each of the depressions 42, 42′. The primers 30, 30′ may be applied as described in reference to FIG. 1B, and thus will graft to the polymeric hydrogel 28, 28′ and not to the surrounding interstitial regions 34, 34′ or enrichment capture site(s) 32, 32′.

In the example shown in FIG. 1C, the enrichment capture site 32, 32′ is a chemical capture agent that is applied on at least some of the interstitial regions 34, 34′. For example, the chemical capture agent may be deposited on at least some of the interstitial regions 34, 34′ using microcontact printing, aerosol printing, etc. to form the enrichment capture site(s) 32, 32′. In still another example, a mask (e.g., a photoresist) may be used to define the space/location where the chemical capture agent will be deposited, and thus where the enrichment capture site 32, 32′ will be formed. The chemical capture agent may then be deposited, and the mask removed (e.g., via lift-off, dissolution, or another suitable technique).

In other examples, the enrichment capture site 32, 32′ is a chemical capture agent that is attached to free functional groups (e.g., those not attached to primers 30, 30′) of the polymeric hydrogel 28, 28′. In still other examples, the enrichment capture site 32, 32′ is a chemical capture agent that is attached to primers that are hybridized to some of the amplification primers 30, 30′. In these examples, the enrichment capture site 32, 32′ will be present in the depressions 42, 42′ and not on the interstitial regions 34, 34′.

Any examples of the chemical capture agent disclosed herein may be used in the example shown in FIG. 1C. The chemical capture agent may be biotin or another member of a binding pair, depending, in part, how the solid support 38 of the target capture beads 36 is functionalized.

As shown in FIG. 1B and FIG. 1C, the substrates 16A and 16A′ or 16B and 16B′ are attached to one another so that the sequencing surfaces 12 and 12′ or 14 and 14′ face each other with the enrichment channel 18 defined therebetween.

The substrates 16A and 16A′ or 16B and 16B′ may be bonded to each other at some of all of the interstitial regions 34, 34′. The bond that is formed between the substrates 16A and 16A′ or 16B and 16B′ may be a chemical bond, or a mechanical bond (e.g., using a fastener, etc.).

Any suitable technique, such as laser bonding, diffusion bonding, anodic bonding, eutectic bonding, plasma activation bonding, glass frit bonding, or other methods known in the art may be used to bond the substrates 16A and 16A′ or 16B and 16B′ together. In an example, a spacer layer (e.g., material 24) may be used to bond the substrates 16A and 16A′ or 16B and 16B′. The spacer layer may be any material 24 that will seal at least some portion of the substrates 16A and 16A′ or 16B and 16B′ together. In some examples, the spacer layer can be a radiation-absorbing material that aids in bonding.

While not shown, it is to be understood that a lid may be bonded to one of the substrate 16A or 16A′ so that the flow cell 10 has one sequencing surface 12 or 14.

### Library Preparation off of the Flow Cell

For the flow cell 10 shown in FIG. 1A, FIG. 1B, and FIG. 1C, preparation of the library fragments may take place outside of the flow cell 10. When library preparation occurs outside of the flow cell 10, any suitable library preparation technique may be used that fragments a longer piece of genetic material and incorporates the desired adapters to the ends of the fragments.

One example of a DNA library preparation technique involves tagmentation on a solid support. In this example, the solid support may be any of the examples set forth herein for the target capture bead 36, and tagged library fragments are immobilized on the surface of the solid support.

In this library preparation example, the solid support has a transposome complexes immobilized thereon, where each transposome complex includes a transposase bound to a first polynucleotide, wherein the first polynucleotide includes (i) a 3′ portion including a transposon end sequence, and (ii) a first tag (adapter). A DNA sample is exposed to the solid support under conditions whereby the DNA sample is fragmented by the transposome complexes, and the 3′ transposon end sequence of the first polynucleotide is transferred to a 5′ end of one strand of the fragments. This produces an immobilized library of double-stranded fragments wherein at least one strand is 5′—tagged with the first tag.

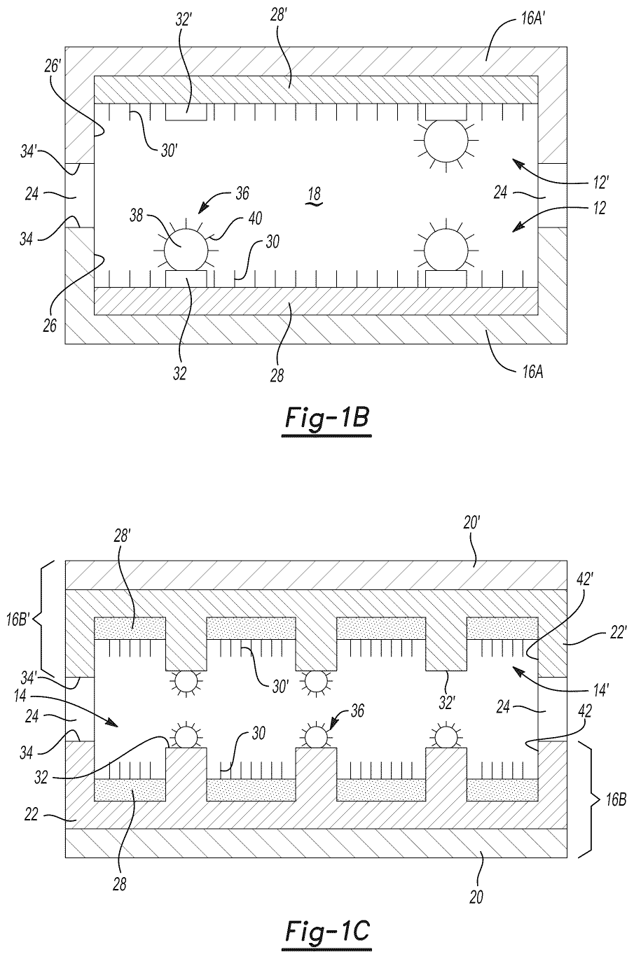

FIG. 2 generally illustrates this library preparation method. In this example, the solid support 38′ includes surface bound transposome complexes 44. The transposome complex 44 includes oligonucleotides (which are grafted to the solid support 38′, and some of which contain the ME sequence) and a transposase enzyme non-covalently bound to the oligonucleotides. The density of these surface bound transposomes 44 can be modulated by varying the density of the grafted oligonucleotides containing the ME sequence or by the amount of transposase added to the solid support 38′. For example, in some embodiments, the transposome complexes 44 are present on the solid support 38′ at a density of at least 10³, 10⁴, 10⁵, or at least 10⁶ complexes per mm².

When a double stranded DNA sample 37 is added to the solid support 38′, the transposome complexes 44 will tagment the added DNA, thus generating double stranded fragments 46 coupled at both ends to the surface of the solid support 38′ (e.g., via the inserted tags). In some examples, the length of bridged fragments 46 can be varied by changing the density of the transposome complexes 44 on the surface. In some examples, the length of the resulting bridged fragments is less than 100 base pairs (bp), 200 bp, 300 bp, 400 bp, 500 bp, 600 bp, 700 bp, 800 bp, 900 bp, 1,000 bp, 1,100 bp, 1,200 bp, 1,300 bp, 1,400 bp, 1,500 bp, 1,600 bp, 1,700 bp, 1,800 bp, 1,900 bp, 2,000 bp, 2,100 bp, 2,200 bp, 2,300 bp, 2,400 bp, 2,500 bp, 2,600 bp, 2,700 bp, 2,800 bp, 2,900 bp, 3,000 bp, 3,100 bp, 3,200 bp, 3,300 bp, 3,400 bp, 3,500 bp, 3,600 bp, 3,700 bp, 3,800 bp, 3,900 bp, 4,000 bp, 4,100 bp, 4,200 bp, 4,300 bp, 4,400 bp, 4,500 bp, 4,600 bp, 4,700 bp, 4,800 bp, 4,900 bp, 5,000 bp, 10,000 bp, 30,000 bp or less than 100,000 bp. In other examples, the length of the resulting bridged fragments 46 is longer. In particular embodiments, the length of the resulting bridged fragments 46 can be within a range defined by an upper and lower limit selected from those exemplified above.

The application of the double stranded DNA sample 37 may involve adding a biological sample to the solid support 38′. The biological sample can be any type that contains DNA and which can be deposited onto the solid surface 38′ for tagmentation. For example, the sample can contain DNA in a variety of states of purification, including purified DNA. However, the sample need not be completely purified, and can contain, for example, DNA mixed with protein, other nucleic acid species, other cellular components and/or any other contaminant. For example, the biological sample may contain a mixture of DNA, protein, other nucleic acid species, other cellular components and/or any other contaminant present in approximately the same proportion as found in vivo. The components may be in the same proportion as found in an intact cell. In some examples, the biological sample has a 260/280 ratio of less than 2.0, 1.9, 1.8, 1.7, 1.6, 1.5, 1.4, 1.3, 1.2, 1.1, 1.0, 0.9, 0.8, 0.7, or less than 0.60. In some embodiments, the biological sample has a 260/280 ratio of at least 2.0, 1.9, 1.8, 1.7, 1.6, 1.5, 1.4, 1.3, 1.2, 1.1, 1.0, 0.9, 0.8, 0.7, or at least 0.60. Because the methods provided herein allow DNA to be bound to a solid support 38′, other contaminants can be removed merely by washing the solid support after surface bound tagmentation occurs. The biological sample can include, for example, a crude cell lysate or whole cells. For example, a crude cell lysate that is applied to a solid support 38′ in this example library preparation method, need not have been subjected to one or more of the separation steps that are traditionally used to isolate nucleic acids from other cellular components.

Thus, in some examples, the biological sample can comprise, for example, blood, plasma, serum, lymph, mucus, sputum, urine, semen, cerebrospinal fluid, bronchial aspirate, feces, and macerated tissue, or a lysate thereof, or any other biological specimen comprising DNA.

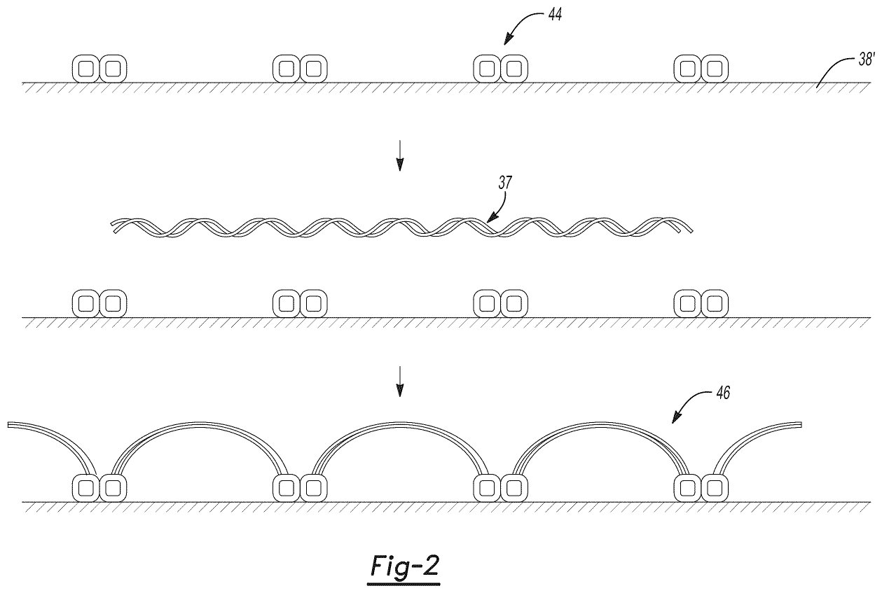

FIGS. 3A and 3B illustrate the tagmentation reaction according to one example. As shown in FIG. 3A, transposomes 44 include a dimer of Tn5 with each monomer 50 binding a double stranded molecule, e.g., the ME adaptor. One strand 48 of the ME adaptor is covalently attached to the surface of a solid support 38′. Transposomes 44 bind the sample DNA and generate two nicks in the DNA backbone, creating fragments 46. In the example shown in FIG. 3A, the nicks are 9 bases apart on either strand. It is to be understood that the transposome 44 can generate a gap of 7, 8, 9, 10, 11, or 12 bp between nicks. One of the two strands (e.g., strand 48) of each ME adaptor is ligated to the 5′ end of each fragment 46 at each nick position. This strand 48 is referred to as “the transferred strand” and is grafted to the surface of the solid support 38′ via its 5′ end. The resulting bridge of the surface tagmentation of FIG. 3A is redrawn in FIG. 3B to clarify the nature of the resulting bridge.

The transferred strand 48 may include a first sequencing primer sequence (e.g., a read 1 sequencing primer sequence) and a first sequence (P5′) that is complementary to at least a portion of one of the amplification primers (e.g., P5) on the flow cell sequencing surface 12, 14. As such, tagmentation introduces one adapter (or tag) to each DNA fragment 46.

After tagmentation, the transposase enzyme may be removed via sodium dodecyl sulfate (SDS) treatment or heat or proteinase K digestion. Removal of the transposase enzymes leaves the fragments 46 attached to the solid support 38′.

The non-transferred strands 48′ of the ME adaptors are adapters that are not incorporated into the DNA fragments 46 during tagmentation, but rather, may be subsequently ligated to the other end of each fragment 46. During tagmentation, the non-transferred strands 48′ may be attached to the transferred strand 48 via base-pairing. In some examples, the non-transferred strand 48′ is an adapter (or tag) including a second sequencing primer sequence (e.g., a read 2 sequencing primer sequence) and a second sequence (P7) that is identical to the at least a portion of another of the amplification primers (P7) on the flow cell sequencing surface 12, 14. During amplification, the identical sequence enables the formation of a copy that is complementary to at least a portion of one of the amplification primers (e.g., P7) on the flow cell sequencing surface 12, 14. As depicted in FIG. 3B, ligation L may be performed to incorporate a non-transferred strand 48′ into each DNA fragment 46 at an end opposite where the transferred strand 48 is incorporated.

While one library preparation technique has been described in detailed, it is to be understood that other library preparation techniques may also be used.

In another library preparation example, one of the adapters (e.g., including a first sequencing primer sequence and a first sequence (P5′) that is complementary to at least a portion of one of the amplification primers (e.g., P5) on the flow cell sequencing surface 12, 14) may be bound to the surface of the solid support 38′.

In this example, a transposome complex may also be bound to the solid support 38′. Prior to loading the transposome complex on the solid support 16, a partial Y-adapter may be mixed with a transposase enzyme (e.g., including two Tn5 molecules) to form an example of the transposome complex. The partial Y-adapter may include two mosaic end sequences that are hybridized to each other. One of the mosaic end sequences has a free end that is able to attach to the fragmented DNA strands 46 during tagmentation, and thus is similar to the transferred strand 48 in FIG. 3A and FIG. 3B. The other of the mosaic end sequences may be attached to a second sequencing primer sequence (e.g., a read 2 sequencing primer sequence), and a second sequence (P7) that has the same sequence as at least a portion of another of the amplification primers (P7) on the flow cell sequencing surface 12, 14, so that its copy is complementary (e.g., P7′) to the amplification primer (P7). In this example, the other of the mosaic end sequences, the second sequencing primer sequence and the second sequence make up an adapter sequence that is not attached to the fragmented DNA strands during tagmentation, and thus are similar to the non-transferred strands 48′ in FIG. 3A and FIG. 3B.

Loading the transposome complex on the solid support 38′ may involve mixing the transposome complex with the solid support 38′, and exposing the mixture to suitable conditions for ligating the mosaic end of the partial Y-adapter to the 3′-end of the adapter sequence on the solid support 38′.

In this example, a tagmentation process may then be performed. During tagmentation, the DNA sample 37 may be applied to the solid support 38′. As the sample contacts the transposome complexes, the longer nucleic acid sample 37 is tagmented. During this example of tagmentation, the sample 36 is fragmented into fragments 64, and each of the fragments 64 is tagged, at its 5′ end, with the free end of the mosaic end of the partial Y-adapter. Successive tagmentation of the longer nucleic acid sample 37 results in a plurality of bridged molecules between the transposome complexes. The bridged molecules wrap around the solid support 38′. The transposase enzyme may be removed, and further extension and ligation is undertaken to ensure sample fragments are attached to the non-attached adapter sequence partial Y-adapter.

In still another library preparation example, a nucleotide library is created in a tube with adapters attached thereto, and then the library is attached to the solid support 38′ through hybridization of the adapters to primers on the solid support 38′. In still another library preparation example, adapter sequences having one member of a binding pair may added to the library fragments in the tube, and then the sequencing-ready library fragments are bound to the solid support 38′ in the tube.

Library preparation that takes place off of the flow cell may also involve a ribonucleic acid (RNA) sample.

In these examples, the RNA sample is first converted to a complementary deoxyribonucleic acid (cDNA) sample. This may be done using reverse transcription, which utilizes a reverse transcriptase enzyme. In some examples, a kit for reverse transcription and second strand synthesis is used. In these examples, the high capacity cDNA reverse transcription kit, from ThermoFisher Scientific, may be used. In other examples, a kit for reverse transcription and template switch (for the second strand) is used. In these examples, the template switching RT enzyme mix, from New England Biolabs, may be used.

Once the double stranded DNA is generated from the RNA sample, any DNA library preparation method, including those described herein, may be used to add adapters/tags to the fragments 46 to generate sequencing-ready nucleic acid fragments attached to the solid support 38′.

### Library Preparation on the Flow Cell

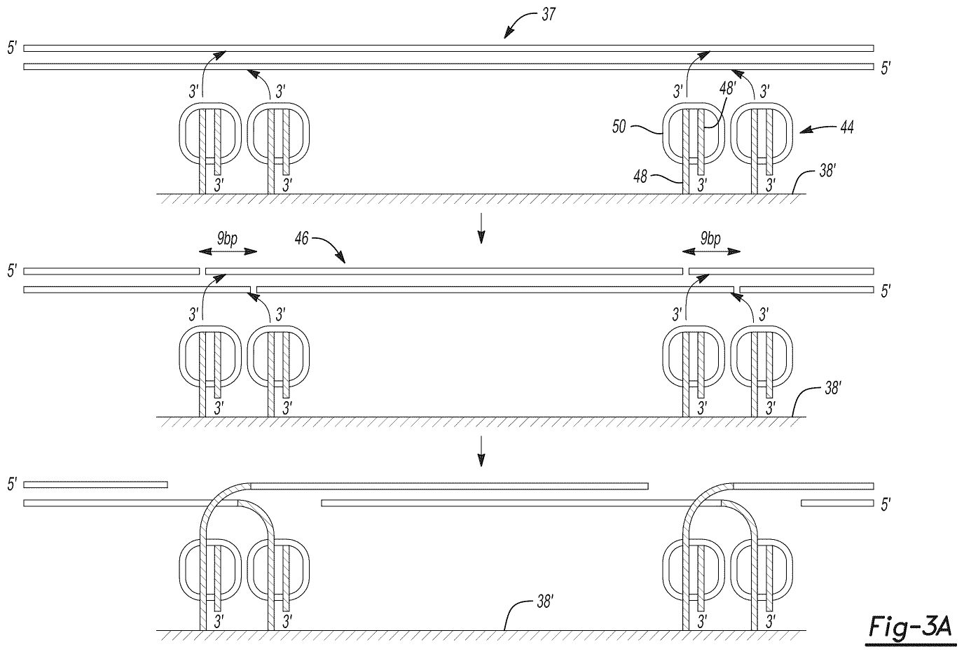

Examples of the flow cell 10 shown in FIG. 1A, FIG. 1B, and FIG. 1C may also be included in a combined library preparation and analysis system 52. This system 52, and the flow cell 10′ included therein, are shown schematically in FIG. 4. The flow cell 10′ includes one or more preparation channels 54, 54′ upstream of the enrichment channel 18. The enrichment channel 18 may be any of the examples disclosed herein for the flow cell 10 that include the sequencing surface 12 or 14 and the enrichment capture sites 32, 32′.

As shown in FIG. 4, the system 52 includes a flow cell reagent system 56 in selective fluid communication with at least the enrichment channel 18. The flow cell reagent system 56 may include reservoirs 58, 60, 62, fluidic channels (e.g., transport channel 64), pumps, valves, and other fluidic device components that can control the flow of reagents to and from the different channels 18, 54, 54′ of the flow cell 10′.

The preparation channels 54, 54′ are utilized for on-flow cell library preparation. When DNA is to be analyzed, the flow cell 10′ may include the DNA library preparation channel 54 upstream of, and in selective fluid communication with the enrichment channel 18. When RNA is to be analyzed, the flow cell 10′ may include both the RNA library preparation channel 54′ and the DNA library preparation channel 54 (which are in fluid communication with each other). In this example, the DNA library preparation channel 54 is upstream of, and in selective fluid communication with the enrichment channel 18; and the RNA library preparation channel 54′ is upstream of, and in selective fluid communication with the DNA library preparation channel 54.

The flow cell 10′ may include any example of the substrate 16, where the preparation channel(s) 54, 54′ is/are defined in a first region and the enrichment channel 18 is defined in a second region. Because they are defined in different regions of the substrate 16, the architecture and component(s) within the channels 54, 54′ and/or 18 may be different.

Similar to the enrichment channel 18, the DNA library preparation channel 54 may be defined between two substrates 16, or between one substrate 16 and a lid. The region of the substrate(s) 16 that defines the DNA library preparation channel(s) 54 may be non-patterned (similar to substrate 16B, 16B′), and may be functionalized to include preparation capture sites (not shown), which temporarily attach the solid supports 38′ used for library preparation.

In one example, the surface(s) of the substrate region(s) defining the DNA library preparation channel 54 is/are coated with a binding pair member (e.g., avidin, streptavidin, biotin, lectin, carbohydrate, nucleic acid binding protein, epitope, antibody, etc.) that is capable of binding to the other binding pair member that is located on the solid support 38′ used for library preparation. Each binding pair member is one preparation capture site. In another example, the surface(s) of the substrate region(s) defining the DNA library preparation channel 54 is/are coated with oligonucleotides (oligos) that have biotinylated oligos hybridized thereto. In this example, the biotinylated oligos are preparation capture sites that are capable of temporarily attaching the solid supports 38′ used for library preparation. In other examples, the preparation capture sites may be capable of any other form of chemical coupling, which can attach the solid support 38′ to the surface(s) of the DNA library preparation channel 54.

DNA library preparation may be performed in the DNA library preparation channel 54, and thus the DNA library preparation channel 54 does not include the amplification primers 30, 30′.

The reservoir 58 is shown as being in selective fluid communication with the DNA library preparation channel 54. This reservoir 58 may include different chambers to selectively deliver a DNA sample 37 and a library preparation fluid to the DNA library preparation channel 54 so that library preparation may take place within the channel 54. In some examples, the library preparation fluid may include library preparation beads (including the solid support 38′ and the transposomes 44 attached thereto as described in reference to FIGS. 2 and 3A). When the DNA sample 37 and the library preparation fluid are introduced into the DNA library preparation channel 54, the solid supports 38′ may bind to the preparation capture sites, and DNA library preparation may take place, for example, using the tagmentation method described herein.

It is to be understood that the reservoir 58 may also deliver different fluids to the DNA library preparation channel 54, depending upon the library preparation method to be used.

Some examples of the flow cell 10′ include the RNA library preparation channel 54′ upstream of the DNA library preparation channel 54.

Similar to the enrichment channel 18 and the DNA library preparation channel 54, the RNA library preparation channel 54′ may be defined between two substrates 16, or between one substrate 16 and a lid. The region of the substrate(s) 16 that defines the RNA library preparation channel(s) 54′ may be non-patterned (similar to substrate 16B, 16B′). The RNA library preparation channel 54′ may be used for the initial preparation of an RNA sample, and thus the RNA library preparation channel 54′ does not include the amplification primers 30, 30′.

The initial process of RNA library preparation may be performed in the RNA library preparation channel 54′. The reservoir 60 is shown as being in selective fluid communication with the RNA library preparation channel 54′. This reservoir 60 may include different chambers to selectively deliver an RNA sample and a library preparation fluid to the RNA library preparation channel 54′ so that RNA may be converted to cDNA within the channel 54′. In some examples, the library preparation fluid may include a reverse transcriptase and reagent(s) for second strand synthesis. In other examples, the library preparation fluid may include a reverse transcriptase and reagent(s) for template switch (for the second strand).

The cDNA prepared in the RNA library preparation channel 54′ may then be delivered (e.g., via a transport channel) to the DNA library preparation channel 54. Within the DNA library preparation channel 54, the cDNA may be tagmented to incorporate adapters (tags) and to generate sequencing ready nucleic acid fragments attached to the solid supports 38 within the DNA library preparation channel 54. Alternatively, any other any suitable DNA library preparation technique may be performed on the cDNA in the DNA library preparation channel 54.

The DNA library preparation channel 54 is in selective fluid communication with the enrichment channel 18. In one example, the transport channel 64 selectively and fluidically connects the preparation channel 54 and the enrichment channel 18, so that the sequencing-ready nucleic acid fragments can be delivered to the enrichment channel 18 for capture, amplification and sequencing.

### Enrichment Methods

An example enrichment method includes preparing library fragments from the genomic deoxyribonucleic acid or the complementary deoxyribonucleic acid; introducing the library fragments to the enrichment channel 18; and incubating the enrichment channel 18 at a predetermined temperature, whereby at least some of the library fragments including the targeted region hybridize to the capture probes 40.

The target capture beads 36 disclosed herein may be pre-attached to the enrichment capture sites 32, 32′ in the flow cell 10, 10′, or may be introduced (e.g., by a user of the flow cell 10, 10′) to the enrichment channel 18 as part of an enrichment and sequencing workflow.

When introduced as part of the sequencing workflow, the target capture beads 36 may be included into the enrichment channel 18 in an enrichment fluid (e.g., a salt buffer containing the target capture beads 36). In the example shown in FIG. 4, the enrichment fluid may be contained within the reservoir 62 that is in selective fluid communication with the enrichment channel 18.

The enrichment fluid may be delivered from the reservoir 62 to the enrichment channel 18. The enrichment capture sites 32, 32′ immobilize some of the target capture beads 36 at the sequencing surface(s) 12, 12′ or 14, 14′ in the enrichment channel 18. It is to be understood that some target capture beads 36 may not attach to the enrichment capture sites 32, 32′, and these target capture beads 36 will be removed from the enrichment channel 18 before further processing. A predetermined time may be allowed to pass before removing the enrichment fluid and any non-immobilized target capture beads 36 from the enrichment channel 18. In an example, the predetermined time may range from about 5 minutes to about 30 minutes in order to obtain a desirable number of immobilized target capture beads 36. Longer incubation times may also be used.

This example method then includes washing away the enrichment fluid and non-trapped target capture beads 36 from the enrichment channel 18. Washing may involve introducing a washing fluid into the enrichment channel 18. The flow may push any target capture beads 36 from that have not become immobilized at the enrichment capture sites 32, 32′ out through an exit port of the enrichment channel 18. The immobilization mechanism (e.g., binding pair, hybridization, covalent bonding, etc.) between the target capture beads 36 and the enrichment capture sites 32, 32′ may prevent any immobilized target capture beads 36 from becoming part of the exit flow.

Whether an off- or on-flow cell library preparation technique is used, the sequencing-ready nucleic acid fragments may be released from the solid supports 38′ prior to being introduced into the enrichment channel 18.

With off-flow cell library preparation techniques, the release of the sequencing-ready nucleic acid fragments from the solid support 38′ may be initiated outside of the flow cell 10, 10′ using any suitable technique. In one example, a cleaving agent may be introduced, and a stimulus may be applied to trigger the cleaving agent to release the sequencing-ready nucleic acid fragments from the solid support 38′. In other examples, the release of the sequencing-ready nucleic acid fragments may involve heating the solid supports 38′ above a melting temperature of a primer that is hybridized to the sequencing-ready nucleic acid fragments. When the sequencing-ready nucleic acid fragments are double stranded (e.g., as shown in FIGS. 2 and 3B), heating may be used to dissociate the fragments from one another. The released sequencing-ready nucleic acid fragments may be separated from the solid supports 38′.

With on-flow cell library preparation techniques, the release of the sequencing-ready nucleic acid fragments from the solid support 38′ may be initiated in the DNA library preparation channel 54 using any suitable technique (e.g., cleaving agent and stimulus, heating, etc.). The solid supports 38′ may remain attached to the preparation capture sites in the DNA library preparation channel 54, and thus the released sequencing-ready nucleic acid fragments may be transported from the DNA library preparation channel 54 into the enrichment channel 18.

Prior to being introduced into the enrichment channel 18, the single-stranded sequencing-ready nucleic acid fragments may have blockers attached to the adapters at each end. The blockers may be selected to reduce adapter-to-primer hybridization. As such, the blockers may at least reduce hybridization of the sequencing-ready nucleic acid fragments to the flow cell primers 30, 30′. In one example, the blockers are XGEN® Universal Blockers available from Integrated DNA Technologies.

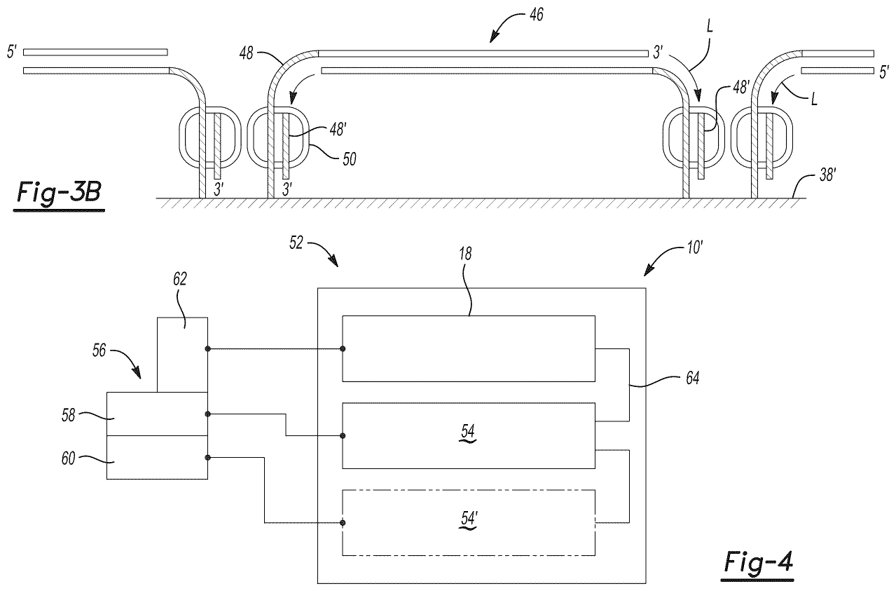

FIG. 5 schematically illustrates the introduction of the end-blocked sequencing-ready nucleic acid fragments 66, 66′ to the enrichment channel 18 having the target capture beads 36 immobilized at the enrichment capture sites 32. Some of the end-blocked sequencing-ready nucleic acid fragments 66′ include the target library fragment, while others of the end-blocked sequencing-ready nucleic acid fragments 66 include a non-target library fragment (e.g., a fragment of the DNA sample that is not of interest for the particular analysis). Because the capture probes 40 have a sequence that is complementary to the target library fragment end-blocked sequencing-ready nucleic acid fragments 66′, at least some of the end-blocked sequencing-ready nucleic acid fragments 66′ respectively hybridize to the capture probes 40. In contrast, the other end-blocked sequencing-ready nucleic acid fragments 66 will not hybridize to the capture probes 40, as the sequences of their non-target library fragments are non-complementary to the capture probes 40. Moreover, the blockers can prevent the end-blocked sequencing-ready nucleic acid fragments 66, 66′ from hybridizing to the flow cell primers 30.

The end-blocked sequencing-ready nucleic acid fragments 66, 66′ may be allowed to incubate in the enrichment channel 18 for a time and at a temperature so that hybridization occurs between the target library fragments and the capture probes 40. The incubation time may range from about 5 minutes to about 2 hours. In an example, the incubation time is about 1.5 hours. The incubation temperature may range from about 50° C. to about 55° C. In an example, the incubation temperature is about 50° C.

In some examples, a crowding reagent may be introduced with the sequencing-ready nucleic acid fragments 66, 66′. Example crowding reagents include dextran sulfate and polyethylene glycol. These crowding reagents can help to crowd the sequencing-ready nucleic acid fragments 66, 66′ to the target capture beads 36.

In other examples, an electric field may be applied to the enrichment channel 18 during the incubation of the sequencing-ready nucleic acid fragments 66, 66′. The electric field may be applied using electrodes integrated into the flow cell 10, 10′ or using external electrodes. The electric field can help to move the sequencing-ready nucleic acid fragments 66, 66′ to the target capture beads 36.

In still other examples, the method may include inducing movement of a fluid containing the sequencing-ready nucleic acid fragments 66, 66′ during the incubation. In one example, the enrichment channel 18 may include brushes, pillars, or other three-dimensional structures on the surface that can facilitate fluid movement.

In yet further examples, electrophoresis may be used to pre-concentrate the sequencing-ready nucleic acid fragments 66, 66′ in the enrichment channel 18. In one example, isotachophoresis (ITP) may be used to speed up hybridization. ITP may be used to concentrate the sequencing-ready nucleic acid fragments 66, 66′ into a narrow band width, e.g., a 1000× higher concentration than the concentration with ITP. This will drive the hybridization kinetics significantly, and thus the hybridization time (e.g., from about 1.5 hours to about 15 minutes).

Other known methods (e.g., adjusting buffer condition, high surface negative charge condition, etc.) for increasing the hybridization efficiency of the sequencing-ready nucleic acid fragments 66′ may also be used.

Any example of the method may then include washing away the end-blocked sequencing-ready nucleic acid fragments 66 from the enrichment channel 18. Washing may involve introducing a washing fluid into the enrichment channel 18. The flow may push any fragments 66 or 66 and 66′ that have not hybridized to capture probes 40 out through an exit port of the enrichment channel 18. The immobilization mechanism (e.g., hybridization) between the end-blocked sequencing-ready nucleic acid fragments 66′ and the capture probes 40 of the target capture beads 36 may prevent any immobilized end-blocked sequencing-ready nucleic acid fragments 66′ from becoming part of the exit flow.

Throughout the enrichment method disclosed herein, it is to be understood that flow rate(s) and/or temperature(s) may be programmed to enhance i) capture probe 40—target library fragment hybridization efficiency and ii) washing processes in order to minimize binding of fragments 66 that include non-target library fragments.

### Library Fragment Release and Sequencing

The immobilized end-blocked sequencing-ready nucleic acid fragments 66′ may then be denatured from the capture probes 40. Denaturation may be accomplished in a seeding reagent (e.g., a salt buffer) and at a suitable denaturing temperature. The denaturing temperature may range from about 70° C. to about 92° C., depending upon the seeding reagent utilized. In an example, the denaturing temperature is about 92° C. The denaturing temperature may be higher than the temperature needed to remove the blockers. As such, denaturing may remove the blockers and an additional removal process would not be utilized. Moreover, because the end-blocked sequencing-ready nucleic acid fragments 66′ are released in the enrichment channel 18, there is minimal to no loss of the released fragments 66′. As such, post-enrichment PCR is not utilized.

The eluted fragments 66′ (each of which includes the target library fragment) are then seeded across the sequencing surface(s) 12, 12′ or 14, 14′. Seeding is accomplished through hybridization between a sequence of the adapters/tags of the fragment 66′ and a complementary one of the primers 30, 30′. Seeding may be performed at a suitable hybridization temperature for the fragment 66′ and the primer(s) 30, 30′.

The location at which the sequencing-ready nucleic acid fragments 66′ seed within the flow cell 10 depends, in part, upon how the primers 30, 30′ are attached. In examples of the flow cell 10 having the non-patterned sequencing surfaces 12, 12′, the released sequencing-ready nucleic acid fragments 66′ will seed across polymeric hydrogels 28, 28′ in the concave regions 26, 26′. In examples of the flow cell 10 having the patterned sequencing surfaces 14, 14′, the released sequencing-ready nucleic acid fragments 66′ will seed across polymeric hydrogels 28, 28′ within each of the depressions 42, 42′.

The solid supports 38 of the target capture beads 36 may then be removed from the enrichment channel 18. Removal of the solid supports 38 may involve any suitable technique, which depends upon the mechanism used to attach the target capture beads 36 to the enrichment capture sites 32, 32′. As examples, denaturing, bond cleaving, etc. may be used.

The seeded sequencing-ready nucleic acid fragments 66′ can then be amplified using cluster generation.

In one example of cluster generation, the sequencing-ready nucleic acid fragments 66′ are copied from the hybridized primers 30, 30′ by 3′ extension using a high-fidelity DNA polymerase. The original sequencing-ready nucleic acid fragments 66′ are denatured, leaving the copies immobilized to the sequencing surfaces 12, 12′ or 14, 14′. The copies include the sequence of the target library fragment. Isothermal bridge amplification or some other form of amplification may be used to amplify the immobilized copies. For example, the copied templates loop over to hybridize to an adjacent, complementary primer 30, 30′, and a polymerase copies the copied templates to form double stranded bridges, which are denatured to form two single stranded strands. These two strands loop over and hybridize to adjacent, complementary primers 30, 30′ and are extended again to form two new double stranded loops. The process is repeated on each template copy by cycles of isothermal denaturation and amplification to create dense clonal clusters. Each cluster of double stranded bridges is denatured. In an example, the reverse strand is removed by specific base cleavage, leaving forward template polynucleotide strands (having the sequence of the target library fragment). Clustering results in the formation of several template polynucleotide strands along the sequencing surfaces 12, 12′ or 14, 14′. This example of clustering is isothermal bridge amplification, and is one example of the amplification that may be performed. It is to be understood that other amplification techniques may be used, such as the exclusion amplification (Examp) workflow (Illumina Inc.).

A sequencing primer may be introduced that hybridizes to a complementary sequence on the template polynucleotide strand. This sequencing primer renders the template polynucleotide strand ready for sequencing. The 3′-ends of the templates and any flow cell-bound primers 30, 30′ (not attached to the copy) may be blocked to prevent interference with the sequencing reaction, and in particular, to prevent undesirable priming.

To initiate sequencing, an incorporation mix may be added to the enrichment channel 18. In one example, the incorporation mix includes a liquid carrier, a polymerase, and fluorescently labeled nucleotides. The fluorescently labeled nucleotides may include a 3′ OH blocking group. When the incorporation mix is introduced into the enrichment channel 18, the fluid enters the channel 18, and in some examples, into the depressions 42, 42′ (where the template polynucleotide strands are present).

The fluorescently labeled nucleotides are added to the sequencing primer (thereby extending the sequencing primer) in a template dependent fashion such that detection of the order and type of nucleotides added to the sequencing primer can be used to determine the sequence of the template. More particularly, one of the nucleotides is incorporated, by a respective polymerase, into a nascent strand that extends the sequencing primer and that is complementary to the template polynucleotide strand. In other words, in at least some of the template polynucleotide strands across the enrichment channel 18, respective polymerases extend the hybridized sequencing primer by one of the nucleotides in the incorporation mix.

The incorporation of the nucleotides can be detected through an imaging event. During an imaging event, an illumination system (not shown) may provide an excitation light to the respective sequencing surfaces 12, 12′ or 14, 14′.

In some examples, the nucleotides can further include a reversible termination property (e.g., the 3′ OH blocking group) that terminates further primer extension once a nucleotide has been added to the sequencing primer. For example, a nucleotide analog having a reversible terminator moiety can be added to the sequencing primer such that subsequent extension cannot occur until a deblocking agent is delivered to remove the moiety. Thus, for examples that use reversible termination, a deblocking reagent can be delivered to the enrichment channel 18 after detection occurs.

Wash(es) may take place between the various fluid delivery steps. The SBS cycle can then be repeated n times to extend the sequencing primer by n nucleotides, thereby detecting a sequence of length n.

In some examples, the forward strands may be sequenced and removed, and then reverse strands are constructed and sequenced as described herein.

While SBS has been described in detail, it is to be understood that the flow cells 10, 10′ described herein may be utilized with other sequencing protocol, for genotyping, or in other chemical and/or biological applications.

### Kits

Some examples disclosed herein are kits. The kits may be used to perform on-flow cell target enrichment disclosed herein.

One example of a kit includes: a library preparation fluid including library preparation beads, each library preparation bead including a first solid support 38′; and a transposome 44 attached to the first solid support 38′; a sample fluid including a genomic deoxyribonucleic acid sequence; and an enrichment fluid including target capture beads 36, each target capture bead 36 including a second solid support 38; and capture probes 40 attached to the second solid support 38, each of the capture probes 40 including a single stranded deoxyribonucleic acid sequence that is complementary to a targeted region of the genomic deoxyribonucleic acid in the sample fluid.

In this example kit, the first solid support 38′ and the second solid support 38 are individually selected from the group consisting of a magnetically responsive material, a glass, a polymer, and a metal.

In this example kit, the capture probes 40 may be attached to the second solid support 38 through a binding pair. Any of the binding pairs disclosed herein may be used.

Some examples of this kit also include a blocking fluid, which includes blocking deoxyribonucleic acid sequences. The blocking fluid may be used to block sequencing-ready nucleic acid fragments 66, 66′ before they are introduced into the enrichment channel 18 of a flow cell 10, 10′.

Another example of a kit includes: an RNA to cDNA conversion sub-kit; and an enrichment fluid including target capture beads 36, each target capture bead 36 including: a solid support 38; and capture probes 40 attached to the solid support 38, each of the capture probes 40 including a single stranded deoxyribonucleic acid sequence that is complementary to a targeted region of cDNA generated using the RNA to cDNA conversion sub-kit.

This example kit may also include a library preparation fluid including library preparation beads, each library preparation bead including a first solid support 38′; and a transposome 44 attached to the first solid support 38′.

In this example kit, the capture probes 40 may be attached to the solid support 38 through a binding pair. Any of the binding pairs disclosed herein may be used.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

In this example, a comparative enrichment method and an example enrichment were performed.

A target region of the PhiX genome was selected and single stranded capture probes with a sequence complementary to the target region were prepared. The target region included genomic coordinate 3194, and the capture probe size was 69 nucleotides. The capture probes were biotinylated at one end.

The NEXTERA™ PhiX library (from Illumina Inc.) was prepared and blockers (from Integrated DNA Technologies) were added to the library. The final concentration of the blocked library was about 2 pM. This library was split in two tubes.

For the comparative enrichment method, the biotinylated capture probes (0.02 μM/1.5 μL/total 1.8E+10 probes) were incubated for about 1.5 hours with the library fragments in one of the tubes to allow hybridization between the library fragments including the target region and the biotinylated capture probes. The tube contents were then exposed to streptavidin beads (M280, 6E+06 beads), where the hybridized probes/fragments became bound to the beads via the biotin label of the capture probes. The beads (with the captured library fragments) were then separated from non-captured library fragments using a magnetic force. The beads (with the captured hybridized probes/fragments) were then loaded on a non-patterned flow cell functionalized with alkyne PEG-biotin and P5/P7 primers. The beads (with the captured hybridized probes/fragments) were immobilized at the alkyne PEG-biotin sites through free streptavidin sites on the beads. The library fragments were eluted off the beads in a saline sodium citrate buffer with sodium dodecyl sulfate at about 92° C. for about 2 minutes, and then the temperature was dropped to about 40° C. for about 5 minutes for seeding.

For the example enrichment method, the biotinylated capture probes (0.02 μM/1.5 μL/total 1.8E+10 probes) were incubated for about 1 (one) hour with the streptavidin beads (M280, 6E+06 beads). The biotinylated capture probes became bound to the beads via the biotin label. The beads (with the captured biotinylated capture probes) were then loaded on a non-patterned flow cell functionalized with alkyne PEG-biotin and P5/P7 primers. The beads (with the captured biotinylated capture probes) were immobilized at the alkyne PEG-biotin sites through free streptavidin sites on the beads. The library fragments in the other of the tubes were loaded onto the flow cell and were allowed to incubate for about 1.5 hours to allow hybridization between the library fragments including the target region and the biotinylated capture probes on the bead surfaces. A washing process was performed to remove non-bound library fragments. The bound library fragments were eluted off the beads in a saline sodium citrate buffer with sodium dodecyl sulfate at about 92° C. for about 2 minutes, and then the temperature was dropped to about 40° C. for about 5 minutes for seeding.

In both the comparative and example methods, the flow cells were washed to remove the beads.

In both the comparative and example methods, clustering was performed using isothermal amplification, and then sequencing was performed. Both the comparative and example methods resulted in a very high number of reads from the target region of the PhiX genome (95% reads within +/−500 bp window, as defined by size of the library).

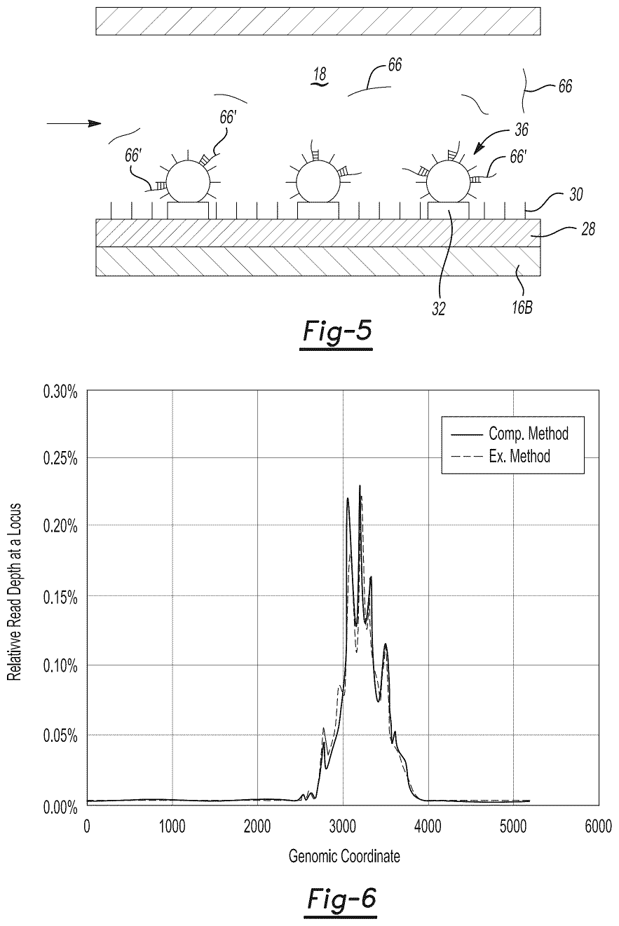

FIG. 6 depicts a relative coverage plot of the clustered target library fragments on the flow cell surface when the comparative enrichment method was used, and on the flow cell surface when the example enrichment method was used. The relative coverage plot is the relative read depth at a locus (%, Y axis) versus the genomic coordinate. The relative coverage plot indicates that the capture probes effectively hybridize the library fragments including the target region, in both the comparative method and the example method. The example method simplifies the workflow (compared to the comparative method) and also achieves target enrichment.

### Example 2

In this example, the target region was from the TRUSIGHT™ Tumor 170 panel (from Illumina Inc.). Single stranded capture probes with a sequence complementary to the target region were prepared. The capture probes were biotinylated at one end.

The biotinylated capture probes were incubated for about 1 (one) hour with the streptavidin beads (M280) to achieve a probe density of about 3.13E+05 capture probes/bead). The biotinylated capture probes became bound to the beads via the biotin label. The beads (with the captured biotinylated capture probes) were then loaded on a non-patterned flow cell functionalized with alkyne PEG-biotin and P5/P7 primers. The beads (with the captured biotinylated capture probes) were immobilized at the alkyne PEG-biotin sites through free streptavidin sites on the beads.

The NEXTERA™ Flex library preparation kit (Illumina Inc.) was used with Human-NA 12878 samples (from Coriell Life Sciences) to prepare the library, and blockers (from Integrated DNA Technologies) were added to the library. The library fragments were loaded onto the flow cell and were allowed to incubate for about 2 hours at about 50° C. to allow hybridization between the library fragments including the target region and the biotinylated capture probes on the surfaces of the immobilized beads.

A washing process was performed to remove non-bound library fragments. The bound library fragments were eluted off the beads in a saline sodium citrate buffer with sodium dodecyl sulfate at about 92° C. for about 2 minutes, and then the temperature was dropped to about 40° C. for about 5 minutes for seeding.

The flow cell was washed to remove the beads, and clustering was performed using isothermal amplification. Sequencing was then performed, and sequencing reads were obtained. The targeted cluster count was determined by dividing the number of reads by 2. The enrichment factor was determined by determining the percentage of the total reads that was aligned to the target region. These results are shown in Table 1 under the sample single library delivery.

This method was repeated, except that the library fragments were loaded onto the flow cell in two shuttles using a rotary valve, and each shuttle was allowed to incubate for about 2 hours at about 50° C. to allow hybridization between the library fragments including the target region and the biotinylated capture probes on the surfaces of the immobilized beads. Washes at about 50° C. were performed between each shuttle delivery and hybridization.

The library fragments were eluted off the beads in a saline sodium citrate buffer with sodium dodecyl sulfate at 92° C. for about 2 minutes, and then the temperature was dropped to about 40° C. for about 5 minutes for seeding.

The flow cell was washed to remove the beads, and clustering was performed using isothermal amplification. Sequencing was then performed, and sequencing reads were obtained. The targeted cluster count and enrichment factor were determined. These results are shown in Table 1 under the sample multiple library delivery.

TABLE 1

| | Input | | Bead | | |
|---|---|---|---|---|---|
| | Library | Probe # | per mm² FC | Targeted | Enrichment |
| Sample | (ng) | per Bead | Surface | clusters | Factor (%) |
| single | 100 | 3.13E+05 | 90,000 | 281,049 | 51.9 |
| library | | | | | |
| delivery | | | | | |
| multiple | 250 | 3.13E+05 | 60,000 | 153,944 | 85 |
| library | | | | | |
| deliveries | | | | | |

As shown in Table 1, while both methods resulted in desirable numbers of clusters from the target region, the multiple delivery method led to an increased enrichment factor.

### Example 3

In this example, the target region was from the TRUSIGHT™ Tumor 170 panel (from Illumina Inc.). Single stranded capture probes with a sequence complementary to the target region were prepared. The capture probes were biotinylated at one end.

The biotinylated capture probes were respectively incubated for about 1 (one) hour with the streptavidin beads (M280) to achieve three different samples with different probe densities−Sample 1=about 5.78E+04 capture probes/bead, Sample 2=about 2.98E+05 capture probes/bead, and Sample 3=about 1.16E+06 capture probes/bead. For each sample, the biotinylated capture probes became bound to the beads via the biotin label. The bead samples (with the captured biotinylated capture probes) were then respectively loaded on three different non-patterned flow cell functionalized with alkyne PEG-biotin and P5/P7 primers. For each sample, the beads (with the captured biotinylated capture probes) were immobilized at the alkyne PEG-biotin sites through free streptavidin sites on the beads.

The NEXTERA™ Flex library preparation kit (Illumina Inc.) was used with Human-NA 12878 samples (from Coriell Life Sciences) to prepare the library, and blockers (from Integrated DNA Technologies) were added to the library. The library fragments were loaded onto the different flow cell and were allowed to incubate for about 2 hours at about 50° C. to allow hybridization between the library fragments including the target region and the biotinylated capture probes on the surfaces of the immobilized beads.

A washing process was performed to remove non-bound library fragments. The bound library fragments were eluted off the beads in a saline sodium citrate buffer with sodium dodecyl sulfate at about 92° C. for about 2 minutes, and then the temperature was dropped to about 40° C. for about 5 minutes for seeding.

The flow cell was washed to remove the beads, and clustering was performed using isothermal amplification. Sequencing was then performed, and sequencing reads were obtained. The targeted cluster count was determined by dividing the number of reads by 2.

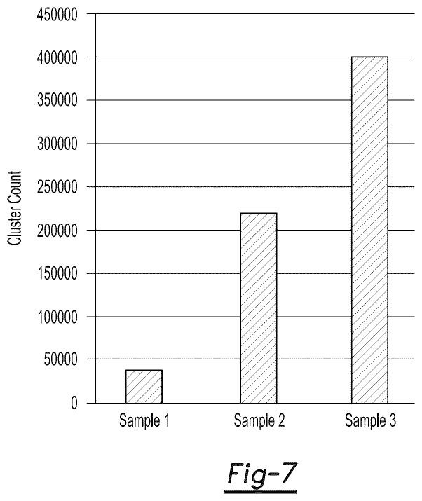

The cluster count for each sample is shown in FIG. 7. As depicted, more of the library fragments with the target region were enriched when the probe density on the beads was increased.

### Additional Notes

Furthermore, it is to be understood that the ranges provided herein include the stated range and any value or sub-range within the stated range, as if they were explicitly recited. For example, a range represented by from about 2 mm to about 300 mm, should be interpreted to include not only the explicitly recited limits of from about 2 mm to about 300 mm, but also to include individual values, such as about 15 mm, 22.5 mm, 245 mm, etc., and sub-ranges, such as from about 20 mm to about 225 mm, etc.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

While several examples have been described in detail, it is to be understood that the disclosed examples may be modified. Therefore, the foregoing description is to be considered non-limiting.

1. A kit, comprising:
a library preparation fluid including library preparation beads, each library preparation bead including:
a first solid support; and
a transposome attached to the first solid support;
a sample fluid including a genomic deoxyribonucleic acid sequence; and
an enrichment fluid including target capture beads, each target capture bead including:
a second solid support; and
capture probes attached to the second solid support, each of the capture probes including a single stranded deoxyribonucleic acid sequence that is complementary to a targeted region of the genomic deoxyribonucleic acid in the sample fluid; and
a system, including:
a substrate;
a preparation channel defined at a first region on the substrate, the preparation channel including preparation capture sites to immobilize the library preparation beads;
an enrichment channel defined at a second region on the substrate downstream of the preparation channel, the enrichment channel including:
amplification primers; and
enrichment capture sites to immobilize the target capture beads; and
a transport channel selectively and fluidically connecting the preparation channel and the enrichment channel.

2. The kit as defined in claim 1, wherein the first solid support and the second solid support are individually selected from the group consisting of a magnetically responsive material, a glass, a polymer, and a metal.

3. The kit as defined in claim 1, wherein the capture probes are attached to the second solid support through a binding pair.

4. The kit as defined in claim 1, further comprising a blocking fluid including blocking deoxyribonucleic acid sequences.

5. The kit as defined in claim 1, wherein a probe density of the target capture beads ranges from about 2*10⁵ capture probes per solid support to about 5*10⁶ capture probes per solid support.

6.-7. (canceled)

8. A system, comprising:
a substrate;
a preparation channel defined at a first region on the substrate, the preparation channel including preparation capture sites to immobilize library preparation beads;
an enrichment channel defined at a second region on the substrate downstream of the preparation channel, the enrichment channel including:
amplification primers; and
enrichment capture sites to immobilize target capture beads; and
a transport channel selectively and fluidically connecting the preparation channel and the enrichment channel.

9. The system as defined in claim 8, wherein the preparation channel does not include the amplification primers.

10. The system as defined in claim 8, further comprising a second substrate attached to the substrate, wherein the second substrate includes:
a second preparation channel including second preparation capture sites; and
a second enrichment channel including second amplification primers and second enrichment capture sites.

11. (canceled)

12. The system as defined in claim 8, further comprising a reagent system in selective fluid communication with the enrichment channel, wherein the reagent system includes:
a reservoir; and
an enrichment fluid contained in the reservoir, the enrichment fluid including target capture beads, each target capture bead including:
a solid support; and
capture probes attached to the solid support, each of the capture probes including a single stranded deoxyribonucleic acid sequence that is complementary to a targeted region of a genomic deoxyribonucleic acid or of a complementary deoxyribonucleic acid.

13. The system as defined in claim 8, further comprising the target capture beads immobilized at the enrichment capture sites, each target capture bead including:
a solid support; and
capture probes attached to the solid support, each of the capture probes including a single stranded deoxyribonucleic acid sequence that is complementary to a targeted region of a genomic deoxyribonucleic acid or of a complementary deoxyribonucleic acid.

14. The system as defined in claim 13, wherein the enrichment capture site includes a first member of a binding pair and the solid support is coated with a second member of the binding pair.

15. The system as defined in claim 8, wherein the enrichment channel includes depressions separated by interstitial regions, and wherein the amplification primers and enrichment capture sites are positioned within each of the depressions.

16.-18. (canceled)

19. A method for using the system of claim 8, comprising:
introducing target capture beads into the enrichment channel whereby at least some of the target capture beads become immobilized at the enrichment capture sites, each of the target capture beads including:
a solid support; and
capture probes attached to the solid support, each of the capture probes including a single stranded nucleic acid sequence that is complementary to a targeted region of a genomic deoxyribonucleic acid or of a complementary deoxyribonucleic acid.

20. The method as defined in claim 19, wherein prior to introducing the target capture beads, the method further comprises preparing the target capture beads by:
synthesizing the capture probes with a first member of a binding pair attached to an end of each capture probe; and
incubating the capture probes with the solid support, wherein the solid support is coated with a second member of the binding pair.

21. The method as defined in claim 20, further comprising controlling a capture probe density of the target capture beads by adjusting a concentration of the capture probes in a mixture used for incubation.

22. The method as defined in claim 19, further comprising:
preparing library fragments from the genomic deoxyribonucleic acid or the complementary deoxyribonucleic acid;
introducing the library fragments to the enrichment channel; and
incubating the enrichment channel at a predetermined temperature, whereby at least some of the library fragments including the targeted region hybridize to the capture probes.

23. The method as defined in claim 22, further comprising blocking ends of the library fragments with blocking oligonucleotides prior to introducing the library fragments into the enrichment channel.

24. The method as defined in claim 22, further comprising introducing a crowding reagent with the library fragments.

25. The method as defined in claim 22, further comprising applying an electric field during the incubation.

26. The method as defined in claim 22, further comprising inducing movement of a fluid containing the library fragment during the incubation.

27. The method as defined in claim 22, further comprising using electrophoresis to pre-concentrate the library fragments in the enrichment channel.
